(19) United States
(12) Patent Application Publication
(10) Pub. No.: US 2025/0331485 A1
(43) Pub. Date: Oct. 30, 2025

(54) SOYBEAN PLANTS HAVING IMPROVED FLAVOR

(71) Applicants: THE CURATORS OF THE UNIVERSITY OF MISSOURI, Columbia, MO (US); THE UNITED STATES OF AMERICA, AS REPRESENTED BY THE SECRETARY OF AGRICULTURE, Washington, DC (US)

(72) Inventors: Kristin D. Bilyeu, Columbia, MO (US); Andrew M. Scaboo, Columbia, MO (US); Bongkosh Vardhanabhuti, Columbia, MO (US)

(21) Appl. No.: 19/192,581

(22) Filed: Apr. 29, 2025

Related U.S. Application Data
(60) Provisional application No. 63/639,979, filed on Apr. 29, 2024.

(51) Int. Cl. A01H 6/54; A01H 1/00; A01H 5/10

(52) U.S. Cl. CPC A01H 6/542; A01H 1/104; A01H 5/10

(57) ABSTRACT

The disclosure relates to soybean genes and mutant alleles thereof associated with improved flavor characteristics. Also disclosed are soybean plants comprising combinations of the mutant alleles along with related methods of improving one or more flavor characteristics of soybean seed.

### CROSS REFERENCE TO RELATED APPLICATIONS

This application claims priority to provisional application U.S. Ser. No. 63/639,979, filed Apr. 29, 2024, which is hereby incorporated herein by reference in its entirety.

### GOVERNMENT SUPPORT

This invention was made with government support under 59-4070-2-003 and 58-5070-2-028 awarded by the Agricultural Research Service. The government has certain rights in the invention.

### SEQUENCE LISTING XML

The instant application contains a sequence listing, which has been submitted in XML file format by electronic submission and is hereby incorporated by reference in its entirety. The XML file, created on Apr. 28, 2025, is named P14768US01.xml and is 64,649 bytes in size.

### TECHNICAL FIELD

The present disclosure relates generally to compositions and methods for identifying, selecting, and producing soybean plants having seeds with improved flavor.

### BACKGROUND

Soybean protein is currently a dominant source for increasing the protein nutrition in human foods and is a major input stock for the alternative protein industry, which is currently formulating new foods that combine functional ingredients, protein nutrition, and target flavor profiles at scale and price competitiveness. Product off-flavors are a major issue for current protein ingredients and soy foods, and soybean off-flavors primarily result from oxidation of polyunsaturated fatty acids (PUFAs). The alternative protein/plant-based protein industry is addressing global sustainable food security and climate resiliency challenges through research and development of next generation foods. A significant impediment to the success of the agriculture system to be responsive to food challenges is the necessity of providing desirable food choices.

The state of the art is commodity soybean seed as an input stock with significant off-flavor issues for protein ingredients and soy foods. An alternative is soybeans having a high protein trait that increases product yield but does not impact flavor. Other options for stocks include lipoxygenase null soybean varieties or high oleic/low linolenic acid (HOLL) oil soybean varieties. However, these traits are not offered together.

Thus, there exists a need in the art for soybeans having improved flavor characteristics including high oleic acid, low linolenic acid, null lipoxygenase enzymes, and/or minimal amounts of PUFAs.

### SUMMARY

Soybean plants, or progeny, plant parts, or plant cells thereof, having one or more improved flavor characteristics are provided. In certain embodiments, the soybean plant comprises a mutant allele of the endogenous FAD2-1A, FAD2-1B, FAD3A, FAD3C, FAD3B, Lox1, Lox2, Lox3, RS2, and/or RS3 gene. In certain embodiments, the soybean plant comprises a mutant allele of the endogenous FAD2-1A, FAD2-1B, FAD3A, FAD3C, Lox1, Lox2, and Lox3 genes. In certain embodiments, the soybean plant comprises a mutant allele of the endogenous FAD2-1A, FAD2-1B, FAD3A, FAD3C, Lox1, Lox2, Lox3, RS2, and RS3 genes. In certain embodiments, the soybean plant comprises a mutant allele of the endogenous FAD2-1A, FAD2-1B, FAD3A, FAD3C, FAD3B, Lox1, Lox2, Lox3, RS2, and RS3 genes.

Methods for improving one or more flavor characteristics of soybean seed are provided. Methods of producing a soybean plant having one or more improved flavor characteristics are also provided. In certain embodiments, the methods comprise introducing a mutant allele of the endogenous FAD2-1A, FAD2-1B, FAD3A, FAD3C, FAD3B, Lox1, Lox2, Lox3, RS2, and/or RS3 gene in a soybean plant. In certain embodiments, this is done through genetic engineering or traditional breeding techniques.

Commodity plant products prepared from the soybean plants, plant parts, and plant cells are also provided.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “of” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

An “allele” is any of one or more alternative forms of a genetic sequence.

A “mutant” or “mutated” allele is an allele comprising at least one mutation relative to the wild-type allele. Mutations are known in the art and may be, for example, frameshift mutations, nonsense mutations, deletions, duplications, substitutions, missense mutations, and insertions. Mutations may result in a “hypomorphic allele,” meaning an allele showing partial loss-of-function. Mutations may alternatively result in a “null allele,” meaning a nonfunctional allele that may result in no gene product or a gene product that does not function properly.

As used herein, a “missense mutation” is a point mutation in which a single nucleotide is changed in a gene sequence, resulting in an amino acid change in the corresponding amino acid.

As used herein, a “nonsense mutation” is a mutation in a sequence of DNA that results in a premature stop codon, or a nonsense codon in the transcribed mRNA, and may result in a truncated protein product.

As used herein, a “frameshift mutation” is a genetic mutation in a polynucleotide sequence caused by insertion or deletion of a number of nucleotides that is not evenly divisible by three. Due to the triplet nature of gene expression by codons, the insertion or deletion can disrupt the reading frame, or the grouping of the codons, resulting in a different translated protein product than from the original non mutated gene.

As used herein, a “deletion” results in the loss of any number of nucleotides e.g. from a single base to an entire gene and surrounding polynucleotide sequences.

As used herein, a “loss of function mutation” is a mutation that renders a protein incapable of carrying out its biological function.

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A “commodity plant product” refers to any composition or product that is comprised of material derived from a plant, seed, plant cell, or plant part of the present disclosure. Commodity plant products may be sold to consumers and can be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, protein products, and any other food for human or animal consumption; biomasses and fuel products; raw material in industry; and beverages, livestock feed, construction material, and starches.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “desired allele”, “targeted allele”, “favorable allele” and “allele of interest” are used interchangeably to refer to an allele associated with a desired trait (e.g., improved flavor characteristics).

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean. An “elite” plant is any plant from an elite line, such that an elite plant is a representative plant from an elite variety.

The term “endogenous” relates to any gene or nucleic acid sequence that is already present in a cell.

The term “expression”, as used herein, generally refers to the production of a functional end-product e.g., an mRNA or a protein (precursor or mature).

As used herein, “gene” includes a nucleic acid fragment that expresses a functional molecule such as, but not limited to, a specific protein coding sequence and regulatory elements, such as those preceding (5′ non-coding sequences) and following (3′ non-coding sequences) the coding sequence.

As used herein, “genome editing” or “gene editing” refers to a type of genetic engineering in which DNA is inserted, replaced, modified, or removed from a genome using artificially engineered nucleases. Examples include but are not limited to use of zinc finger nucleases (ZFNs), TAL effector nucleases (TALENs), meganucleases, CRISPR/Cas9, and other CRISPR related technologies.

As used herein, a “genetic map” is a description of genetic linkage relationships among loci on one or more chromosomes within a given species, generally depicted in a diagrammatic or tabular form. For each genetic map, distances between loci are measured by the recombination frequencies between them. Recombination events between loci can be detected using a variety of markers. A genetic map is a product of the mapping population, types of markers used, and the polymorphic potential of each marker between different populations. The order and genetic distances between loci can differ from one genetic map to another.

A “genetic locus” as used herein generally refers to the location on a chromosome of the plant where a gene is found.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm may refer to seeds, cells (including protoplasts and calli) or tissues from which new plants may be grown, as well as plant parts that can be cultured into a whole plant (e.g., stems, buds, roots, leaves, etc.).

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

As used herein, the term “hybrid” refers to a seed and/or plant produced when at least two genetically dissimilar parents are crossed. An F1 hybrid is the first filial generation of offspring of two genetically distinct parents.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the terms “include,” “includes,” and “including” are to be construed as at least having the features to which they refer while not excluding any additional unspecified features.

As used herein, the terms “introducing”, “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele (in the heterozygous state), with the result being that the desired allele becomes fixed in the desired genetic background after at least one round of selfing.

As used herein, an “isolated” nucleic acid molecule is substantially separated away from other nucleic acid sequences with which the nucleic acid is normally associated, such as, from the chromosomal or extrachromosomal DNA of a cell in which the nucleic acid naturally occurs. The term also embraces nucleic acids that are biochemically purified so as to substantially remove contaminating nucleic acids and other cellular components.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript.

Modified plants or seeds contain molecular changes in their genetic materials, including either genetic or epigenetic modifications. Typically, modified plants or seeds, or a parental or progenitor line thereof, have a natural genetic variation or have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In certain embodiments, a modified plant provided herein comprises no non-plant genetic material or sequences. In yet another embodiment, a modified plant provided herein comprises no interspecies genetic material or sequences.

A “non-naturally occurring variety of soybean” is any variety of soybean that does not naturally exist in nature. A “non-naturally occurring variety of soybean” may be produced by any method known in the art, including, but not limited to, transforming a soybean plant or germplasm, transfecting a soybean plant or germplasm and crossing a naturally occurring variety of soybean with a non-naturally occurring variety of soybean. In certain embodiments, a “non-naturally occurring variety of soybean” may comprise one of more heterologous nucleotide sequences. In certain embodiments, a “non-naturally occurring variety of soybean” may comprise one or more non-naturally occurring copies of a naturally occurring nucleotide sequence (i.e., extraneous copies of a gene that naturally occurs in soybean). In certain embodiments, a “non-naturally occurring variety of soybean” may comprise a non-natural combination of two or more naturally occurring nucleotide sequences (i.e., two or more naturally occurring genes that do not naturally occur in the same soybean).

As used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide.

By “operably linked” or “operably associated,” it is meant that the indicated elements are functionally related to each other, and are also generally physically related. Thus, the term “operably linked” or “operably associated” as used herein, refers to nucleotide sequences on a single nucleic acid molecule that are functionally associated. Therefore, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of the nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

As used herein, “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant.

As used herein, “plant part” includes any part of a plant, such as a plant organ, a plant cell, a plant protoplast, a plant cell tissue culture or a tissue culture from which a whole plant can be regenerated, a plant cell that is intact in a plant, a clone, a micropropagation, plant callus, a plant cell clump, a plant transplant, a vegetative propagation, a pod, a part of a pod, a leaf, a part of a leaf, pollen, an ovule, an embryo, a petiole, a shoot or a part thereof, a stem or a part thereof, a root or a part thereof, a root tip, a cutting, a seed, a part of a seed, a hypocotyl, a cotyledon, a scion, a graft, a stock, a rootstock, pericarp, a pistil, an anther, or a flower. Seed can be mature or immature. Pollen or ovules may be viable or non-viable. Also, any developmental stage is included, such as seedlings, cuttings prior or after rooting, mature plants or leaves.

The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

The terms “polypeptide,” “peptide”, and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., Madison, Wis.), and MUSCLE (Edgar, “MUSCLE: multiple sequence alignment with high accuracy and high throughput” Nucleic Acids Research 32(5):1792-7 (2004)) for instance with default parameters. The BLAST program set to the default parameters, available from the National Center for Biotechnology Information (NCBI), can also be used to obtain an optimal alignment of protein or nucleic acid sequences and to calculate the percentage of sequence identity. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

Soybean Genes and Mutant Alleles Thereof Associated with Improved Flavor Characteristics

Mutant alleles and mutant allele combinations associated with improved flavor characteristics in soybean are provided. In certain embodiments, the mutant allele is an allele of one or more of the endogenous FAD2-1A, FAD2-1B, FAD3A, FAD3C, FAD3B, Lox1, Lox2, Lox3, RS2, and/or RS3 genes.

The endogenous FAD2-1A gene (Glyma.10g278000) is located at nucleotides 50,013,483 to 50,015,460 of chromosome 10 of the Glycine max Williams 82 genome assembly version Wm82.a2.v1. In certain embodiments, the endogenous FAD2-1A gene comprises a nucleotide sequence having at least 90%, at least 95%, at least 96%, at least 97%, at least 98%, at least 99%, or 100% sequence identity to SEQ ID NO: 1. In certain embodiments, the endogenous FAD2-1A gene encodes a polypeptide having at least 90%, at least 95%, at least 96%, at least 97%, at least 98%, at least 99%, or 100% sequence identity to SEQ ID NO: 11. In certain embodiments, the mutant allele of the endogenous FAD2-1A gene comprises a missense mutation. In certain embodiments, the missense mutation is a serine to asparagine substitution at position 117 (S117N). In certain embodiments, the mutant allele of the endogenous FAD2-1A gene comprises a frameshift mutation. There are small functional consequences to the effect on fatty acid profiles from these alleles, with the frameshift mutation having more significant effects than the missense mutation. The endogenous FAD2-1B gene (Glyma.20G111000) is located at nucleotides 35,315,629 to 35,319,063 of chromosome 20 of the Glycine max Williams 82 genome assembly version Wm82.a2.v1. In certain embodiments, the endogenous FAD2-1B gene comprises a nucleotide sequence having at least 90%, at least 95%, at least 96%, at least 97%, at least 98%, at least 99%, or 100% sequence identity to SEQ ID NO: 2. In certain embodiments, the endogenous FAD2-1B gene encodes a polypeptide having at least 90%, at least 95%, at least 96%, at least 97%, at least 98%, at least 99%, or 100% sequence identity to SEQ ID NO: 12. In certain embodiments, the mutant allele of the endogenous FAD2-1B gene comprises a missense mutation. In certain embodiments, the missense mutation is a proline to arginine substitution at position 137 (P137R). In certain embodiments, the missense mutation is an isoleucine to threonine substitution at position 143 (I143T). The FAD2-1A and FAD2-1B genes have been described in, for example, U.S. Pat. Nos. 9,035,129; 9,198,365; 10,087,454; 10,329,576; and 10,774,337, each of which is herein incorporated by reference.

The endogenous FAD3A gene (Glyma.14G194300) is located at nucleotides 45,935,667 to 45,939,896 of chromosome 14 of the Glycine max Williams 82 genome assembly version Wm82.a2.v1. In certain embodiments, the endogenous FAD3A gene comprises a nucleotide sequence having at least 90%, at least 95%, at least 96%, at least 97%, at least 98%, at least 99%, or 100% sequence identity to SEQ ID NO: 3. In certain embodiments, the endogenous FAD3A gene encodes a polypeptide having at least 90%, at least 95%, at least 96%, at least 97%, at least 98%, at least 99%, or 100% sequence identity to SEQ ID NO: 13. In certain embodiments, the mutant allele of the endogenous FAD3A gene comprises a splice site mutation. The endogenous FAD3C gene (Glyma.18G062000) is located at nucleotides 5,646,501 to 5,649,337 of chromosome 18 of the Glycine max Williams 82 genome assembly version Wm82.a2.v1. In certain embodiments, the endogenous FAD3C gene comprises a nucleotide sequence having at least 80%, at least 90%, at least 95%, at least 96%, at least 97%, at least 98%, at least 99%, or 100% sequence identity to SEQ ID NO: 4. In certain embodiments, the endogenous FAD3C gene encodes a polypeptide having at least 90%, at least 95%, at least 96%, at least 97%, at least 98%, at least 99%, or 100% sequence identity to SEQ ID NO: 14. In certain embodiments, the mutant allele of the endogenous FAD3C gene comprises a missense mutation. In certain embodiments, the missense mutation is a glycine to glutamic acid substitution at position 128 (G128E). In certain embodiments, the missense mutation is a histidine to tyrosine substitution at position 304 (H304Y). The endogenous FAD3B gene (Glyma.02G227200) is located at nucleotides 41,419,655 to 41,423,881 of chromosome 2 of the Glycine max Williams 82 genome assembly version Wm82.a2.v1. In certain embodiments, the endogenous FAD3B gene comprises a nucleotide sequence having at least 90%, at least 95%, at least 96%, at least 97%, at least 98%, at least 99%, or 100% sequence identity to SEQ ID NO: 5. In certain embodiments, the endogenous FAD3B gene encodes a polypeptide having at least 90%, at least 95%, at least 96%, at least 97%, at least 98%, at least 99%, or 100% sequence identity to SEQ ID NO: 15. In certain embodiments, the mutant allele of the endogenous FAD3B gene comprises a splice site mutation.

The endogenous Lox1 gene (Glyma.13G347600) is located at nucleotides 43,769,020 to 43,773,290 of chromosome 13 of the Glycine max Williams 82 genome assembly version Wm82.a2.v1. In certain embodiments, the endogenous Lox1 gene comprises a nucleotide sequence having at least 90%, at least 95%, at least 96%, at least 97%, at least 98%, at least 99%, or 100% sequence identity to SEQ ID NO: 6. In certain embodiments, the endogenous Lox1 gene encodes a polypeptide having at least 90%, at least 95%, at least 96%, at least 97%, at least 98%, at least 99%, or 100% sequence identity to SEQ ID NO: 16. In certain embodiments, the mutant allele of the endogenous Lox1 gene comprises a frameshift mutation. The endogenous Lox2 gene (Glyma.13G347500) is located at nucleotides 43,761,726 to 43,766,023 of chromosome 13 of the Glycine max Williams 82 genome assembly version Wm82.a2.v1. In certain embodiments, the endogenous Lox2 gene comprises a nucleotide sequence having at least 90%, at least 95%, at least 96%, at least 97%, at least 98%, at least 99%, or 100% sequence identity to SEQ ID NO: 7. In certain embodiments, the endogenous Lox2 gene encodes a polypeptide having at least 90%, at least 95%, at least 96%, at least 97%, at least 98%, at least 99%, or 100% sequence identity to SEQ ID NO: 17. In certain embodiments, the mutant allele of the endogenous Lox2 gene comprises a missense mutation. In certain embodiments, the missense mutation is a histidine to glutamine substitution at position 532 (H532Q). The endogenous Lox3 gene (Glyma.15G026300) is located at nucleotides 2,123,753 to 2,128,104 of chromosome 15 of the Glycine max Williams 82 genome assembly version Wm82.a2.v1. In certain embodiments, the endogenous Lox3 gene comprises a nucleotide sequence having at least 90%, at least 95%, at least 96%, at least 97%, at least 98%, at least 99%, or 100% sequence identity to SEQ ID NO: 8. In certain embodiments, the endogenous Lox3 gene encodes a polypeptide having at least 90%, at least 95%, at least 96%, at least 97%, at least 98%, at least 99%, or 100% sequence identity to SEQ ID NO: 18. In certain embodiments, the mutant allele of the endogenous Lox3 gene comprises a frameshift mutation.

The endogenous RS2 gene (Glyma.06G179200) is located at nucleotides 15,217,418 to 15,223,877 of chromosome 6 of the Glycine max Williams 82 genome assembly version Wm82.a2.v1. In certain embodiments, the endogenous RS2 gene comprises a nucleotide sequence having at least 90%, at least 95%, at least 96%, at least 97%, at least 98%, at least 99%, or 100% sequence identity to SEQ ID NO: 9. In certain embodiments, the endogenous RS2 gene encodes a polypeptide having at least 90%, at least 95%, at least 96%, at least 97%, at least 98%, at least 99%, or 100% sequence identity to SEQ ID NO: 19. In certain embodiments, the mutant allele of the endogenous RS2 gene comprises an in-frame deletion. In certain embodiments, the mutant allele of the endogenous RS2 gene comprises a deletion of the tryptophan at position 360 (W360del). In certain embodiments, the mutant allele of the endogenous RS2 gene comprises a frameshift mutation. The endogenous RS3 gene (Glyma.05G003900) is located at nucleotides 307,460 to 312,091 of chromosome 5 of the Glycine max Williams 82 genome assembly version Wm82.a2.v1. In certain embodiments, the endogenous RS3 gene comprises a nucleotide sequence having at least 90%, at least 95%, at least 96%, at least 97%, at least 98%, at least 99%, or 100% sequence identity to SEQ ID NO: 10. In certain embodiments, the endogenous RS3 gene encodes a polypeptide having at least 90%, at least 95%, at least 96%, at least 97%, at least 98%, at least 99%, or 100% sequence identity to SEQ ID NO: 20. In certain embodiments, the mutant allele of the endogenous RS3 gene comprises a missense mutation. In certain embodiments, the missense mutation is a glycine to glutamic acid substitution at position 75 (G75E). The RS2 and RS3 genes have been described in, for example, U.S. Pat. Nos. 8,471,107 and 10,081,814, each of which is herein incorporated by reference.

Additional mutant alleles of the endogenous FAD2-1A, FAD2-1B, FAD3A, FAD3C, FAD3B, Lox1, Lox2, Lox3, RS2, and RS3 genes suitable for use in the present disclosure include, but are not limited to, those disclosed in the Soykb Soybean Allele Catalog Tool (soykb.org/SoybeanAlleleCatalogTool). Mutant alleles of the endogenous FAD2-1A, FAD2-1B, FAD3A, FAD3C, FAD3B, Lox1, Lox2, Lox3, RS2, and RS3 genes suitable for use in the present disclosure also include those disclosed in Bilyeu et al., (2006) Crop Sci. 46:1913-1918; Chappell & Bilyeu, (2006) Plant Breeding 125:535-536; Pham et al., (2013) Mol. Breeding 33:895-907; and Lenis et al., (2010) Theor. Appl. Genet. 120:1139-1149, each of which is herein incorporated by reference.

### Reducing Expression or Activity of a Soybean Gene

Several embodiments of the disclosure relate to reducing expression or activity of a gene of the disclosure (e.g., FAD2-1A, FAD2-1B, FAD3A, FAD3C, FAD3B, Lox1, Lox2, Lox3, RS2, and/or RS3) in a soybean plant. As used herein “reduced,” “reduction,” or the like refers to any detectable decrease in an experimental group (e.g., a soybean plant with a mutant allele described herein) as compared to a control group (e.g., wild-type soybean plant that does not comprise the mutant allele). Methods for reducing expression or activity of genes or gene products are well documented in the art.

In certain embodiments of the present disclosure, the expression or activity of the gene of the disclosure is decreased or eliminated by disrupting the gene. The gene of the disclosure may be disrupted by any method known in the art, for example, by genome editing, transposon tagging, or mutagenizing plants using random or targeted mutagenesis and optionally selecting for plants that have decreased expression or activity.

In certain embodiments, the gene of the disclosure (e.g., FAD2-1A, FAD2-1B, FAD3A, FAD3C, FAD3B, Lox1, Lox2, Lox3, RS2, and/or RS3) is modified using genome editing technology. Targeted modification of plant genomes through the use of genome editing methods can be used to reduce expression of a gene through modification of plant genomic DNA. Genome editing methods can enable targeted insertion of one or more nucleic acids of interest into a plant genome. Genome editing uses engineered nucleases such as RNA guided DNA endonucleases or nucleases composed of sequence specific DNA binding domains fused to a non-specific DNA cleavage module. These engineered nucleases enable efficient and precise genetic modifications by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

“Targeted DNA modification” can be used synonymously with targeted DNA mutation and refers to the introduction of a site specification modification that alters or changes the nucleotide sequence at a specific genomic locus of the soybean plant.

The targeted DNA modification described herein may be any modification known in the art such as, for example, insertion, deletion, or single nucleotide polymorphism (SNP). Additionally, the targeted DNA modification in the genomic locus may be located anywhere in the genomic locus, such as, for example, a coding region of the encoded polypeptide (e.g., exon), a non-coding region (e.g., intron), a regulatory element, or untranslated region.

The type and location of the targeted DNA modification of the gene is not particularly limited so long as the targeted DNA modification results in reduced expression or activity of the protein encoded by the gene. In certain embodiments, the targeted DNA modification is a deletion of one or more nucleotides, preferably contiguous, of the genomic locus.

In certain embodiments, a reduction in the expression or activity of the protein encoded by the gene is due to a targeted DNA modification at a genomic locus of a plant that results in one or more of the following: (a) reduced expression of the gene; (b) reduced transcriptional activity of the protein encoded by the gene; (c) generation of one or more alternatively spliced transcripts of the gene; (d) frameshift mutation in one or more exons of the gene; (e) deletion of a substantial portion of the gene or deletion of the full open reading frame of the gene; (f) repression of an enhancer motif present within a regulatory region encoding the gene; or (g) modification of one or more nucleotides or deletion of a regulatory element operably linked to the expression of the gene wherein the regulatory element is present within a promoter, intron, 3′UTR, terminator or a combination thereof.

In certain embodiments, the genomic locus has more than one (e.g., 2, 3, 4, 5, 6, 7, 8, 9, or 10) targeted DNA modification. For example, the translated region and a regulatory element of a genomic locus may each comprise a targeted DNA modification. In certain embodiments, the plant may have targeted DNA modifications at more than one (e.g., 2, 3, 4, 5, 6, 7, 8, 9, or 10) genomic loci comprising a gene of the disclosure (e.g., FAD2-1A, FAD2-1B, FAD3A, FAD3C, FAD3B, Lox1, Lox2, Lox3, RS2, and/or RS3).

The targeted DNA modification of the genomic locus may be done using any genome modification technique known in the art. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas-gRNA systems (based on bacterial CRISPR-Cas systems), guided cpf1 endonuclease systems, and the like. In certain embodiments, the introduction of a DSB can be combined with the introduction of a polynucleotide modification template.

A polynucleotide modification template can be introduced into a cell by any method known in the art, such as, but not limited to, transient introduction methods, transfection, electroporation, microinjection, particle mediated delivery, topical application, whiskers mediated delivery, delivery via cell-penetrating peptides, or mesoporous silica nanoparticle (MSN)-mediated direct delivery.

The polynucleotide modification template can be introduced into a cell as a single stranded polynucleotide molecule, a double stranded polynucleotide molecule, or as part of a circular DNA (vector DNA). The polynucleotide modification template can also be tethered to the guide RNA and/or the Cas endonuclease. Tethered DNAs can allow for co-localizing target and template DNA, useful in genome editing and targeted genome regulation, and can also be useful in targeting post-mitotic cells where function of endogenous HR machinery is expected to be highly diminished (Mali et al. 2013 Nature Methods Vol. 10: 957-963.) The polynucleotide modification template may be present transiently in the cell or it can be introduced via a viral replicon.

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

TAL effector nucleases (TALEN) are a class of sequence-specific nucleases that can be used to make double-strand breaks at specific target sequences in the genome of a plant or other organism. (Miller et al. (2011) Nature Biotechnology 29:143-148).

A TALEN comprises a TAL effector DNA binding domain and an endonuclease domain. TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AhvI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or Pl- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, WO2016007347, published on Jan. 14, 2016, and WO201625131, published on Feb. 18, 2016, all of which are incorporated by reference herein.

The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing, binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure include, for example a Cas9 protein, a Cas12a protein, a Cas12b protein, or complexes of these.

As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein the guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference).

A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference.

Other Cas endonuclease systems have been described in PCT patent applications PCT/US16/32073, filed May 12, 2016 and PCT/US16/32028 filed May 12, 2016, both applications incorporated herein by reference.

“Cas9” (formerly referred to as Cas5, Csn1, or Csx12) herein refers to a Cas endonuclease of a type II CRISPR system that forms a complex with a crNucleotide and a tracrNucleotide, or with a single guide polynucleotide, for specifically recognizing and cleaving all or part of a DNA target sequence. Cas9 protein comprises a RuvC nuclease domain and an HNH (H-N-H) nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). A type II CRISPR system includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA.

Any guided endonuclease can be used in the methods disclosed herein. Such endonucleases include, but are not limited to Cas9, Cas12a, and Cas12b endonucleases. Many endonucleases have been described to date that can recognize specific PAM sequences (see for example—Jinek et al. (2012) Science 337 p 816-821, PCT patent applications PCT/US16/32073, filed May 12, 2016 and PCT/US16/32028 filed May 12, 2016 and Zetsche B et al. 2015. Cell 163, 1013) and cleave the target DNA at a specific position. It is understood that based on the methods and embodiments described herein utilizing a guided Cas system one can now tailor these methods such that they can utilize any guided endonuclease system.

The guide polynucleotide can also be a single molecule (also referred to as single guide polynucleotide) comprising a crRNA sequence linked to a tracrRNA sequence. The single guide polynucleotide comprises a first nucleotide sequence domain (referred to as Variable Targeting domain or VT domain) that can hybridize to a nucleotide sequence in a target DNA and a Cas endonuclease recognition domain (CER domain), that interacts with a Cas endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crRNA and the tracrRNA may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas endonuclease, wherein the guide polynucleotide/Cas endonuclease complex (also referred to as a guide polynucleotide/Cas endonuclease system) can direct the Cas endonuclease to a genomic target site, enabling the Cas endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

The term “variable targeting domain” or “VT domain” is used interchangeably herein and includes a nucleotide sequence that can hybridize (is complementary) to one strand (nucleotide sequence) of a double strand DNA target site. In certain embodiments, the variable targeting domain comprises a contiguous stretch of 12 to 30 nucleotides. The variable targeting domain can be composed of a DNA sequence, an RNA sequence, a modified DNA sequence, a modified RNA sequence, or any combination thereof.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas system that can form a complex with a type II Cas endonuclease, wherein the guide RNA/Cas endonuclease complex can direct the Cas endonuclease to a DNA target site, enabling the Cas endonuclease to recognize, bind to, and optionally nick or cleave (introduce a single or double strand break) the DNA target site.

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein the guide RNA/Cas endonuclease complex can direct the Cas endonuclease to a DNA target site, enabling the Cas endonuclease to recognize, bind to, and optionally nick or cleave (introduce a single or double strand break) the DNA target site. A guide RNA/Cas endonuclease complex herein can comprise Cas protein(s) and suitable RNA component(s) of any of the four known CRISPR systems (Horvath and Barrangou, 2010, Science 327:167-170) such as a type I, II, or III CRISPR system. A guide RNA/Cas endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide of the methods and compositions described herein may be any polynucleotide sequence that targets the genomic loci of a plant cell comprising a polynucleotide that encodes an amino acid sequence that has at least 80%, at least 90%, at least 95%, at least 96%, at least 97%, at least 98%, or at least 99% identical to SEQ ID NO: 1, 2, 3, 4, 5, 6, 7, 8, 9, or 10. In certain embodiments, the guide polynucleotide is a guide RNA. The guide polynucleotide may also be present in a recombinant DNA construct.

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in the cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in WO2016025131, published on Feb. 18, 2016, incorporated herein in its entirety by reference.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by a Cas endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB), can prompt NHEJ or HDR processes which can lead to modifications at the target site.

A targeting method herein can be performed in such a way that two or more DNA target sites are targeted in the method, for example. Such a method can optionally be characterized as a multiplex method. In certain embodiments, two, three, four, five, six, seven, eight, nine, ten, or more target sites can be targeted at the same time. A multiplex method is typically performed by a targeting method herein in which multiple different RNA components are provided, each designed to direct a guide polynucleotide/Cas endonuclease complex to a unique DNA target site.

The guide polynucleotide/Cas endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

Various methods and compositions can be employed to obtain a cell or organism having a polynucleotide of interest inserted in a target site. Such methods can employ homologous recombination to provide integration of the polynucleotide of Interest at the target site. In one method provided, a polynucleotide of interest is provided to the organism cell in a donor DNA construct. As used herein, “donor DNA” is a DNA construct that comprises a polynucleotide of Interest to be inserted into the target site. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

The region of homology on the donor DNA can have homology to any sequence flanking the target site. While in certain embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In certain embodiments, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology.

Further uses for guide RNA/Cas endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

In certain embodiments, transposon tagging is used to decrease or eliminate the activity of the gene of the disclosure (e.g., FAD2-1A, FAD2-1B, FAD3A, FAD3C, FAD3B, Lox1, Lox2, Lox3, RS2, and/or RS3). Transposon tagging comprises inserting a transposon within an endogenous gene in the pathway to decrease or eliminate expression. In certain embodiments, the expression is decreased or eliminated by inserting a transposon within a regulatory region or coding region of the gene encoding the polypeptide. A transposon that is within an exon, intron, 5′ or 3′ untranslated sequence, a promoter or any other regulatory sequence of a gene may be used to decrease or eliminate the expression and/or activity of the encoded polypeptide.

Methods for the transposon tagging of specific genes in plants are well known in the art. See, e.g., Maes et al., (1999) Trends Plant Sci. 4:90-96; Dharmapuri & Sonti, (1999) FEMS Microbiol. Lett. 179:53-59; Meissner et al., (2000) Plant J. 22:265-74; Phogat et al., (2000) J. Biosci. 25:57-63; Walbot, (2000) Curr. Opin. Plant Biol. 2:103-07; Gai et al., (2000) Nucleic Acids Res. 28:94-96; Fitzmaurice et al., (1999) Genetics 153:1919-28. In addition, the TUSC process for selecting Mu insertions in selected genes has been described in Bensen et al., (1995) Plant Cell 7:75-84; Mena et al., (1996) Science 274:1537-40; and U.S. Pat. No. 5,962,764, each of which is herein incorporated by reference.

Additional methods for reducing or eliminating the expression of endogenous genes in plants are also known in the art and may be similarly applied to the present disclosure. These methods include other forms of mutagenesis, such as ethyl methanesulfonate-induced mutagenesis, deletion mutagenesis, and fast neutron deletion mutagenesis used in a reverse genetics sense (with PCR) to identify plant lines, in which the endogenous gene has been mutated or deleted. For examples of these methods see Ohshima et al., (1998) Virology 243:472-81; Okubara et al., (1994) Genetics 137:867-74; and Quesada et al., (2000) Genetics 154:421-36, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See McCallum et al., (2000) Nat. Biotechnol. 18:455-57, herein incorporated by reference.

Mutations may impact gene expression or interfere with the activity of an encoded polypeptide. Insertional mutations in gene exons usually result in null alleles. Mutations in conserved residues are particularly effective in inhibiting the activity of the encoded protein. Conserved residues of polypeptides suitable for mutagenesis with the goal to eliminate activity have been described. Such mutants may be isolated according to well-known procedures and mutations in different target gene loci may be stacked by genetic crossing. See, e.g., Gruis et al., (2002) Plant Cell 14:2863-82.

In certain embodiments, dominant mutants are used to trigger RNA silencing due to gene inversion and recombination of a duplicated gene locus. See, e.g., Kusaba et al., (2003) Plant Cell 15:1455-67.

The disclosure encompasses additional methods for decreasing or eliminating the activity of one or more target polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984, each of which are herein incorporated by reference. See also WO 1998/49350, WO 1999/07865, WO 1999/25821, and Beetham et al., (1999) Proc. Natl. Acad. Sci. USA 96:8774-78, each of which is herein incorporated by reference.

In certain embodiments, reducing the expression or activity of the gene of the disclosure (e.g., FAD2-1A, FAD2-1B, FAD3A, FAD3C, FAD3B, Lox1, Lox2, Lox3, RS2, and/or RS3) comprises introducing into a plant or plant cell a silencing element, thereby reducing or eliminating the level or expression of a polynucleotide or a polypeptide encoded by the gene.

As used herein, “silencing element” refers to a polynucleotide that is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. A single polynucleotide employed in the methods can comprise one or more silencing elements to the same or different target polynucleotides. The silencing element can be produced in vivo (i.e., in a host cell such as a plant) or in vitro.

Non-limiting examples of silencing elements include, a sense suppression element, an antisense suppression element, a double stranded RNA, a siRNA, an amiRNA, a miRNA, or a hairpin suppression element. Non-limiting examples of silencing elements that can be employed to decrease expression of these target sequences or additionally sequences targeting genes involved in recombination comprise fragments and variants of the sense or antisense sequence or consists of the sense or antisense sequence of wild type polynucleotide or polypeptide sequences, variant polynucleotides, variant polypeptides, cognate promoter sequences, ortholog sequences, variants or fragments thereof. The silencing element can further comprise additional sequences that advantageously effect transcription and/or the stability of a resulting transcript. For example, the silencing elements can comprise at least one thymine residue at the 3′ end. This can aid in stabilization. Thus, the silencing elements can have at least 1, 2, 3, 4, 5, 6, 7, 8, 9, 10 or more thymine residues at the 3′ end. Enhancer suppressor elements can also be employed in conjunction with the silencing elements.

In certain embodiments, introducing the silencing element reduces the polynucleotide level and/or the polypeptide level of the target sequence to less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control.

In certain embodiments, decreasing expression of the gene of the disclosure (e.g., FAD2-1A, FAD2-1B, FAD3A, FAD3C, FAD3B, Lox1, Lox2, Lox3, RS2, and/or RS3) is obtained by sense suppression or cosuppression. For cosuppression, an expression cassette is designed to express an RNA molecule corresponding to all or part of a messenger RNA encoding a polypeptide in the “sense” orientation. Over expression of the RNA molecule may result in decreased expression of the native gene. Accordingly, multiple plant lines transformed with the cosuppression expression cassette are screened to identify those that show the desired degree of inhibition of polypeptide expression.

Typically, a sense suppression element has substantial sequence identity to the target polynucleotide, typically greater than about 65% sequence identity, greater than about 85% sequence identity, about 90%, 91%, 92%, 93%, 94%, 95%, 96%, 97%, 98% or 99% sequence identity. See, U.S. Pat. Nos. 5,283,184 and 5,034,323; herein incorporated by reference. The sense suppression element can be any length so long as it allows for the suppression of the targeted sequence. In other implementations, the sense suppression element is, for example, 15, 16, 17, 18 19, 20, 22, 25, 30, 50, 100, 150, 200, 250, 300, 350, 400, 450, 500, 600, 700, 900, 1000, 1100, 1200, 1300 nucleotides or longer of the target polynucleotides. In other implementations, the sense suppression element is, for example, about 15-25, 25-100, 100-150, 150-200, 200-250, 250-300, 300-350, 350-400, 450-500, 500-550, 550-600, 600-650, 650-700, 700-750, 750-800, 800-850, 850-900, 900-950, 950-1000, 1000-1050, 1050-1100, 1100-1200, 1200-1300, 1300-1400, 1400-1500, 1500-1600, 1600-1700, 1700-1800 nucleotides or longer of the target polynucleotides.

The polynucleotide used for cosuppression may correspond to all or part of the sequence encoding the polypeptide, all or part of the 5′ and/or 3′ untranslated region of a polypeptide transcript or all or part of both the coding sequence and the untranslated regions of a transcript encoding a polypeptide. In certain embodiments where the polynucleotide comprises all or part of the coding region for the polypeptide, the expression cassette is designed to eliminate the start codon of the polynucleotide so that no protein product will be translated.

Any region of the target polynucleotide can be used to design the domain of the silencing element that shares sufficient sequence identity to allow expression of the hairpin transcript to decrease the level of the target polynucleotide. For instance, the domain can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof. In specific embodiments, a domain of the silencing element shares sufficient homology to at least about 15, 16, 17, 18, 19, 20, 22, 25 or 30 consecutive nucleotides from about nucleotides 1-50, 25-75, 75-125, 50-100, 125-175, 175-225, 100-150, 150-200, 200-250, 225-275, 275-325, 250-300, 325-375, 375-425, 300-350, 350-400, 425-475, 400-450, 475-525, 450-500, 525-575, 575-625, 550-600, 625-675, 675-725, 600-650, 625-675, 675-725, 650-700, 725-825, 825-875, 750-800, 875-925, 925-975, 850-900, 925-975, 975-1025, 950-1000, 1000-1050, 1025-1075, 1075-1125, 1050-1100, 1125-1175, 1100-1200, 1175-1225, 1225-1275, 1200-1300, 1325-1375, 1375-1425, 1300-1400, 1425-1475, 1475-1525, 1400-1500, 1525-1575, 1575-1625, 1625-1675, 1675-1725, 1725-1775, 1775-1825, 1825-1875, 1875-1925, 1925-1975, 1975-2025, 2025-2075, 2075-2125, 2125-2175, 2175-2225, 1500-1600, 1600-1700, 1700-1800, 1800-1900, 1900-2000 of the target sequence.

### Expression Constructs

As used herein, the term “expression construct” refers to a combination of nucleic acid sequences that provides for transcription of an operably linked nucleic acid sequence. A gene of the disclosure (e.g., FAD2-1A, FAD2-1B, FAD3A, FAD3C, FAD3B, Lox1, Lox2, Lox3, RS2, and/or RS3) can be provided in an expression construct. It is further recognized that various other expression constructs are also described herein. For example, expression constructs encoding a silencing element, an RNA-guided endonuclease, or other genome editing molecules are described herein. One of skill will understand how to apply the disclosure to any expression construct.

Expression constructs generally include regulatory elements that are functional in the intended host cell in which the expression construct is to be expressed. Thus, a person of ordinary skill in the art can select regulatory elements for use in bacterial host cells, yeast host cells, plant host cells, insect host cells, mammalian host cells, and human host cells. Regulatory elements include promoters, transcription termination sequences, translation termination sequences, enhancers, and polyadenylation elements.

An expression construct can comprise a promoter sequence operably linked to a polynucleotide sequence as described herein. Promoters can be incorporated into a polynucleotide using standard techniques known in the art. Multiple copies of promoters or multiple promoters can be used in an expression construct as described herein. In certain embodiments, a promoter can be positioned about the same distance from the transcription start site in the expression construct as it is from the transcription start site in its natural genetic environment. Some variation in this distance is permitted without substantial decrease in promoter activity. A transcription start site is typically included in the expression construct.

The choice of promoter will vary depending on the temporal and spatial requirements for expression, and also depending on the host cell to be transformed. Thus, for example, expression of the nucleotide sequences can be in any plant and/or plant part, (e.g., in leaves, in stems, in inflorescences, in roots, seeds and/or seedlings, and the like). In many cases, however, expression in multiple tissues is desirable. Although many promoters from dicotyledons have been shown to be operational in monocotyledons and vice versa, a dicotyledonous promoter may be selected for expression in dicotyledons, and a monocotyledonous promoter for expression in monocotyledons. However, there is no restriction to the provenance of selected promoters; it is sufficient that they are operational in driving the expression of the nucleotide sequences in the desired cell.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, zein promoters including maize zein promoters, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-1a promoter, ubiquitin promoter, actin promoter, alcA gene promoter, pin2 promoter (Xu et al., 1993), maize Wipl promoter, maize trpA gene promoter (U.S. Pat. No. 5,625,136), maize CDPK gene promoter, and RUBISCO SSU promoter (U.S. Pat. No. 5,034,322) can also be used. Constitutive promoters (such as the CaMV, ubiquitin, actin, or NOS promoter), developmentally-regulated promoters, and inducible promoters (such as those promoters than can be induced by heat, light, hormones, or chemicals) are also contemplated for use with polynucleotide expression constructs described herein. These various types of promoters are known in the art.

Examples of constitutive promoters include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences in transgenic plants, especially monocotyledons. Further, the promoter expression cassettes described by McElroy et al. (Mol. Gen. Genet. 231: 150-160 (1991)) can be easily modified for the expression of the nucleotide sequences and are particularly suitable for use in monocotyledonous hosts.

In certain embodiments, tissue specific/tissue preferred promoters can be used. Tissue specific or preferred expression patterns include, but are not limited to, green tissue specific or preferred, root specific or preferred, stem specific or preferred, and flower specific or preferred. Promoters suitable for expression in green tissue include many that regulate genes involved in photosynthesis and many of these have been cloned from both monocotyledons and dicotyledons. Non-limiting examples of tissue-specific promoters include those associated with genes encoding the seed storage proteins (such as 0-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters include the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; and the cestrum yellow leaf curling virus promoter disclosed in WO 01/73087, all incorporated by reference.

Additional examples of tissue-specific/tissue preferred promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612). In some particular embodiments, the nucleotide sequences are operatively associated with a root-preferred promoter.

Promoters useful for seed-specific expression include the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters include but are not limited to the S-E9 small subunit RuBP carboxylase promoter and the Kunitz trypsin inhibitor gene promoter (Kti3).

In certain embodiments, inducible promoters can be used. Thus, for example, chemical-regulated promoters can be used to modulate the expression of a gene in a plant through the application of an exogenous chemical regulator. Regulation of the expression of nucleotide sequences via promoters that are chemically regulated enables the polypeptides to be synthesized only when the crop plants are treated with the inducing chemicals. Depending upon the objective, the promoter may be a chemical-inducible promoter, where application of a chemical induces gene expression, or a chemical-repressible promoter, where application of the chemical represses gene expression.

Chemical inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid (e.g., the PR1a system), steroid steroid-responsive promoters (see, e.g., the glucocorticoid-inducible promoter in Schena et al. (1991) Proc. Natl. Acad. Sci. USA 88, 10421-10425 and McNellis et al. (1998) Plant J. 14, 247-257) and tetracycline-inducible and tetracycline-repressible promoters (see, e.g., Gatz et al. (1991) Mol. Gen. Genet. 227, 229-237, and U.S. Pat. Nos. 5,814,618 and 5,789,156, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters.

Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), and the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421). Also included are the benzene sulphonamide-inducible (U.S. Pat. No. 5,364,780) and alcohol-inducible (Int'l Patent Application Publication Nos. WO 97/06269 and WO 97/06268) systems and glutathione 5-transferase promoters. Likewise, one can use any of the inducible promoters described in Gatz (1996) Current Opinion Biotechnol. 7:168-172 and Gatz (1997) Annu. Rev. Plant Physiol. Plant Mol. Biol. 48:89-108. Other chemically inducible promoters useful for directing the expression of the nucleotide sequences in plants are disclosed in U.S. Pat. No. 5,614,395 herein incorporated by reference in its entirety. Chemical induction of gene expression is also detailed in the published application EP 0 332 104 (to Ciba-Geigy) and U.S. Pat. No. 5,614,395. In certain embodiments, a promoter for chemical induction can be the tobacco PR-1a promoter.

A number of non-translated leader sequences derived from viruses are known to enhance gene expression. Specifically, leader sequences from Tobacco Mosaic Virus (TMV, the “ω-sequence”), Maize Chlorotic Mottle Virus (MCMV) and Alfalfa Mosaic Virus (AMV) have been shown to be effective in enhancing expression (Gallie et al. (1987) Nucleic Acids Res. 15:8693-8711; and Skuzeski et al. (1990) Plant Mol. Biol. 15:65-79). Other leader sequences known in the art include, but are not limited to, picornavirus leaders such as an encephalomyocarditis (EMCV) 5′ noncoding region leader (Elroy-Stein et al. (1989) Proc. Natl. Acad. Sci. USA 86:6126-6130); potyvirus leaders such as a Tobacco Etch Virus (TEV) leader (Allison et al. (1986) Virology 154:9-20); Maize Dwarf Mosaic Virus (MDMV) leader (Allison et al. (1986), supra); human immunoglobulin heavy-chain binding protein (BiP) leader (Macejak & Samow (1991) Nature 353:90-94); untranslated leader from the coat protein mRNA of AMV (AMV RNA 4; Jobling & Gehrke (1987) Nature 325:622-625); tobacco mosaic TMV leader (Gallie et al. (1989) Molecular Biology of RNA 237-256); and MCMV leader (Lommel et al. (1991) Virology 81:382-385). See also, Della-Cioppa et al. (1987) Plant Physiol. 84:965-968.

Expression constructs may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides described herein. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

An expression construct can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. Of course, many examples of suitable selectable markers are known in the art and can be used in the expression cassettes described herein.

Examples of selectable markers include, but are not limited to, a nucleotide sequence encoding neo or nptII, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette.

Additional selectable markers include, but are not limited to, a nucleotide sequence encoding β-glucuronidase or uidA (GUS) that encodes an enzyme for which various chromogenic substrates are known; an R-locus nucleotide sequence that encodes a product that regulates the production of anthocyanin pigments (red color) in plant tissues (Dellaporta et al., “Molecular cloning of the maize R-nj allele by transposon-tagging with Ac,” pp. 263-282 In: Chromosome Structure and Function: Impact of New Concepts, 18th Stadler Genetics Symposium (Gustafson & Appels eds., Plenum Press 1988)); a nucleotide sequence encoding β-lactamase, an enzyme for which various chromogenic substrates are known (e.g., PADAC, a chromogenic cephalosporin) (Sutcliffe (1978) Proc. Natl. Acad. Sci. USA 75:3737-3741); a nucleotide sequence encoding xylE that encodes a catechol dioxygenase (Zukowsky et al. (1983) Proc. Natl. Acad. Sci. USA 80:1101-1105); a nucleotide sequence encoding tyrosinase, an enzyme capable of oxidizing tyrosine to DOPA and dopaquinone, which in turn condenses to form melanin (Katz et al. (1983) J. Gen. Microbiol. 129:2703-2714); a nucleotide sequence encoding β-galactosidase, an enzyme for which there are chromogenic substrates; a nucleotide sequence encoding luciferase (lux) that allows for bioluminescence detection (Ow et al. (1986) Science 234:856-859); a nucleotide sequence encoding aequorin, which may be employed in calcium-sensitive bioluminescence detection (Prasher et al. (1985) Biochem. Biophys. Res. Comm. 126:1259-1268); or a nucleotide sequence encoding green fluorescent protein (Niedz et al. (1995) Plant Cell Reports 14:403-406). One of skill in the art is capable of choosing a suitable selectable marker for use in an expression cassette.

Optionally the polynucleotide is codon optimized to remove features inimical to expression and codon usage is optimized for expression in the particular crop (see, for example, U.S. Pat. No. 6,051,760; EP 0359472; EP 80385962; EP 0431829; and Perlak et al. (1991) PNAS USA 88:3324-3328; all of which are herein incorporated by reference).

### Transformation Methods

Several embodiments relate to plant cells, plant tissues, plants, and seeds that comprise a recombinant DNA (e.g., a genome editing molecule, a silencing element) as described herein.

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Transformation of a cell may be stable or transient. Thus, in certain embodiments, a plant cell is stably transformed with a nucleic acid molecule. In other embodiments, a plant is transiently transformed with a nucleic acid molecule. “Transient transformation” in the context of a polynucleotide means that a polynucleotide is introduced into the cell and does not integrate into the genome of the cell. By “stably introducing” or “stably introduced” in the context of a polynucleotide introduced into a cell is intended the introduced polynucleotide is stably incorporated into the genome of the cell, and thus the cell is stably transformed with the polynucleotide.

“Stable transformation” or “stably transformed” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

In certain embodiments, transformation of a cell comprises nuclear transformation. In other embodiments, transformation of a cell comprises plastid transformation (e.g., chloroplast transformation).

Procedures for transforming plants are well known and routine in the art and are described throughout the literature. Non-limiting examples of methods for transformation of plants include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Agrobacterium-mediated transformation is a commonly used method for transforming plants, in particular, dicot plants, because of its high efficiency of transformation and because of its broad utility with many different species. Agrobacterium-mediated transformation typically involves transfer of the binary vector carrying the foreign DNA of interest to an appropriate Agrobacterium strain that may depend on the complement of vir genes carried by the host Agrobacterium strain either on a co-resident Ti plasmid or chromosomally (Uknes et al. (1993) Plant Cell 5:159-169). The transfer of the recombinant binary vector to Agrobacterium can be accomplished by a triparental mating procedure using Escherichia coli carrying the recombinant binary vector, a helper E. coli strain that carries a plasmid that is able to mobilize the recombinant binary vector to the target Agrobacterium strain. Alternatively, the recombinant binary vector can be transferred to Agrobacterium by nucleic acid transformation (Höfgen & Willmitzer (1988) Nucleic Acids Res. 16:9877).

Transformation of a plant by recombinant Agrobacterium usually involves co-cultivation of the Agrobacterium with explants from the plant and follows methods well known in the art. Transformed tissue is regenerated on selection medium carrying an antibiotic or herbicide resistance marker between the binary plasmid T-DNA borders.

Another method for transforming plants, plant parts and/or plant cells involves propelling inert or biologically active particles at plant tissues and cells. See, e.g., U.S. Pat. Nos. 4,945,050; 5,036,006 and 5,100,792. Generally, this method involves propelling inert or biologically active particles at the plant cells under conditions effective to penetrate the outer surface of the cell and afford incorporation within the interior thereof. When inert particles are utilized, the vector can be introduced into the cell by coating the particles with the vector containing the nucleic acid of interest. Alternatively, a cell or cells can be surrounded by the vector so that the vector is carried into the cell by the wake of the particle. Biologically active particles (e.g., dried yeast cells, dried bacterium or a bacteriophage, each containing one or more nucleic acids sought to be introduced) also can be propelled into plant tissue.

Thus, in particular embodiments, a plant cell can be transformed by any method known in the art and as described herein and intact plants can be regenerated from these transformed cells using any of a variety of known techniques. Plant regeneration from plant cells, plant tissue culture and/or cultured protoplasts is described, for example, in Evans et al. (Handbook of Plant Cell Cultures, Vol. 1, MacMilan Publishing Co. New York (1983)); and Vasil I. R. (ed.) (Cell Culture and Somatic Cell Genetics of Plants, Acad. Press, Orlando, Vol. I (1984), and Vol. II (1986)). Methods of selecting for transformed transgenic plants, plant cells and/or plant tissue culture are routine in the art and can be employed in the methods provided herein.

Likewise, the genetic properties engineered into the transgenic seeds and plants, plant parts, and/or plant cells described above can be passed on by sexual reproduction or vegetative growth and therefore can be maintained and propagated in progeny plants. Generally, maintenance and propagation make use of known agricultural methods developed to fit specific purposes such as harvesting, sowing or tilling.

A nucleotide sequence therefore can be introduced into the plant, plant part and/or plant cell in any number of ways that are well known in the art. The methods do not depend on a particular method for introducing one or more nucleotide sequences into a plant, only that they gain access to the interior of at least one cell of the plant. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, in separate transformation events, or, for example, in plants, as part of a breeding protocol.

Soybean Plants with Improved Flavor Characteristics

Several embodiments relate to soybean plant cells, plant tissues, plants, and seeds having improved flavor characteristics. Certain embodiments encompass a progeny or a descendant of a soybean plant with improved flavor characteristics, as well as seeds derived from the plants with improved flavor characteristics and cells derived from the plants with improved flavor characteristics as described herein.

In certain embodiments, the one or more improved flavor characteristics comprise high oleic acid and/or low linolenic acid relative to a control soybean plant without the mutant allele. In certain embodiments, a soybean plant, plant part, or plant product (e.g., oil) that has “high oleic acid” content has an oleic acid content of about 70%, 75%, 76%, 77%, 78%, 79%, 80%, 81%, 82%, 83%, or 84% to about 85%, 86%, 87%, 88%, 89%, or 90% (of total fatty acids) by weight. In certain embodiments, the soybean plant, plant part, or plant product (e.g., oil) has an oleic acid content of at least 75%, at least 80%, at least 81%, at least 82%, at least 83%, at least 84%, or at least 85% (of total fatty acids) by weight. In certain embodiments, a soybean plant, plant part, or plant product (e.g., oil) that has “low linolenic acid” content has a linolenic acid content of about 0.1%, 0.2%, 0.3%, 0.4%, 0.5%, 0.6%, 0.7%, 0.8%, 0.9%, or 1% to about 2%, 2.5%, 3%, 4%, or 5% (of total fatty acids) by weight. In certain embodiments, the soybean plant, plant part, or plant product (e.g., oil) has a linolenic acid content of less than 5%, less than 4%, less than 3%, less than 2%, or less than 1% (of total fatty acids) by weight.

In certain embodiments, plant cells of the present disclosure are capable of regenerating a plant or plant part. In certain embodiments, plant cells are not capable of regenerating a plant or plant part. Examples of cells not capable of regenerating a plant include, but are not limited to, endosperm, seed coat (testa and pericarp), and root cap.

Several embodiments provide a commodity plant product prepared from the plants with improved flavor characteristics. In certain embodiments, examples of plant products include, without limitation, grain, oil, protein, and meal. In certain embodiments, a commodity plant product is plant grain (e.g., grain suitable for use as feed or for processing), plant oil (e.g., oil suitable for use as food or biodiesel), flakes, white flakes, or plant meal (e.g., meal suitable for use as feed). Several embodiments provide methods for producing a commodity plant product comprising processing the soybean plant or plant part of the disclosure to obtain the product. In certain embodiments, the methods comprise isolating, extracting, and/or preparing a protein composition (e.g., soy protein composition, soy protein concentrate (SPC), soy protein isolate (SPI), soy flour, white flake, textured soy protein (TSP) from the soybean plant or plant part, such as from seed.

The product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g. by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extend or sequentially. Generally, the plants are grown for some time before the product is produced.

In certain embodiments, the soybean plant is a non-naturally occurring variety of soybean. In certain embodiments, the soybean plant is at least about 50%, 55%, 60%, 65%, 70%, 75%, 80%, 85%, 90%, 95%, 96%, 97%, 98, 99%, or 100% identical to that of an elite variety of soybean.

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity, and plant height is desirable. Traditional plant breeding is an important tool in developing new and improved commercial crops. This disclosure encompasses methods for producing a plant by crossing a first parent plant with a second parent plant. In some embodiments, one or both of the parent plants is a plant displaying a phenotype as described herein. In some embodiments, each parent donates a complementary subset of the desired alleles.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Detection Tools

Several embodiments provide a method for identifying a soybean plant having one or more improved flavor characteristics, or cells or tissues thereof. In certain embodiments, the method includes using primers or probes which specifically recognize a portion of the sequence of a gene of the disclosure (e.g., FAD2-1A, FAD2-1B, FAD3A, FAD3C, FAD3B, Lox1, Lox2, Lox3, RS2, and/or RS3). Probes and primers are provided which are of sufficient nucleotide length to bind specifically to the target DNA sequence under the reaction or hybridization conditions. Suitable probes and primers are at least 10, 11, 12, 13, 14, 15, 16, 17, 18, 19, 20, 21, 22, 23, 24, 25, 26, 27, 28, 29 or 30 nucleotides in length, and less than 35, 34, 33, 32, 31, 30, 29, 28, 27, 26, 2, 5 2, 423, 22, 21, 20, 19, 18, 17, 16, 15, 14, 13, or 12 nucleotides in length. Such probes and primers can hybridize specifically to a target sequence under high stringency hybridization conditions. In certain embodiments, probes and primers have complete or 100% DNA sequence similarity of contiguous nucleotides with the target sequence, although probes which differ from the target DNA sequence but retain the ability to hybridize to target DNA sequence may also be used. Reverse complements of the primers and probes disclosed herein are also provided and can be used in the methods and compositions described herein.

Several embodiments provide kits for identifying plants having one or more improved flavor characteristics, the kits comprising at least one oligonucleotide primer or probe that specifically recognizes a gene of the disclosure (e.g., FAD2-1A, FAD2-1B, FAD3A, FAD3C, FAD3B, Lox1, Lox2, Lox3, RS2, and/or RS3). In certain embodiments, at least one oligonucleotide comprises a detectable label. The kit may also include one or more positive or negative controls.

### EMBODIMENTS

The following numbered embodiments also form part of the present disclosure:
1. A soybean plant, or a progeny, a plant part, or a plant cell thereof, having one or more improved flavor characteristics, comprising a mutant allele of the endogenous FAD2-1 A, FAD2-1B, FAD3A, FAD3C, FAD3B, Lox1, Lox2, Lox3, RS2, and/or RS3 gene.
2. The soybean plant of embodiment 1, wherein the plant comprises a mutant allele of the endogenous FAD2-1A, FAD2-1B, FAD3A, FAD3C, Lox1, Lox2, and Lox3 genes.
3. The soybean plant of embodiment 1, wherein the plant comprises a mutant allele of the endogenous FAD2-1A, FAD2-1B, FAD3A, FAD3C, Lox1, Lox2, Lox3, RS2, and RS3 genes.
4. The soybean plant of embodiment 1, wherein the plant comprises a mutant allele of the endogenous FAD2-1A, FAD2-1B, FAD3A, FAD3C, FAD3B, Lox1, Lox2, Lox3, RS2, and RS3 genes.
5. The soybean plant of any one of embodiments 1-5, wherein the mutant allele is a loss-of-function allele.
6. The soybean plant of any one of embodiments 1-5, wherein the mutant allele is a null allele or a hypomorphic allele.
7. The soybean plant of any one of embodiments 1-6, wherein the endogenous FAD2-1A gene comprises a nucleotide sequence having at least 90%, at least 95%, at least 96%, at least 97%, at least 98%, at least 99%, or 100% sequence identity to SEQ ID NO: 1, wherein the endogenous FAD2-1B gene comprises a nucleotide sequence having at least 90%, at least 95%, at least 96%, at least 97%, at least 98%, at least 99%, or 100% sequence identity to SEQ ID NO: 2, wherein the endogenous FAD3A gene comprises a nucleotide sequence having at least 90%, at least 95%, at least 96%, at least 97%, at least 98%, at least 99%, or 100% sequence identity to SEQ ID NO: 3, wherein the endogenous FAD3C gene comprises a nucleotide sequence having at least 80%, at least 90%, at least 95%, at least 96%, at least 97%, at least 98%, at least 99%, or 100% sequence identity to SEQ ID NO: 4, wherein the endogenous FAD3B gene comprises a nucleotide sequence having at least 90%, at least 95%, at least 96%, at least 97%, at least 98%, at least 99%, or 100% sequence identity to SEQ ID NO: 5, wherein the endogenous Lox1 gene comprises a nucleotide sequence having at least 90%, at least 95%, at least 96%, at least 97%, at least 98%, at least 99%, or 100% sequence identity to SEQ ID NO: 6, wherein the endogenous Lox2 gene comprises a nucleotide sequence having at least 90%, at least 95%, at least 96%, at least 97%, at least 98%, at least 99%, or 100% sequence identity to SEQ ID NO: 7, wherein the endogenous Lox3 gene comprises a nucleotide sequence having at least 90%, at least 95%, at least 96%, at least 97%, at least 98%, at least 99%, or 100% sequence identity to SEQ ID NO: 8, wherein the endogenous RS2 gene comprises a nucleotide sequence having at least 90%, at least 95%, at least 96%, at least 97%, at least 98%, at least 99%, or 100% sequence identity to SEQ ID NO: 9, or wherein the endogenous RS3 gene comprises a nucleotide sequence having at least 90%, at least 95%, at least 96%, at least 97%, at least 98%, at least 99%, or 100% sequence identity to SEQ ID NO: 10.
8. The soybean plant of any one of embodiments 1-7, wherein the endogenous FAD2-1A gene encodes a polypeptide having at least 90%, at least 95%, at least 96%, at least 97%, at least 98%, at least 99%, or 100% sequence identity to SEQ ID NO: 11, wherein the endogenous FAD2-1B gene encodes a polypeptide having at least 90%, at least 95%, at least 96%, at least 97%, at least 98%, at least 99%, or 100% sequence identity to SEQ ID NO: 12, wherein the endogenous FAD3A gene encodes a polypeptide having at least 90%, at least 95%, at least 96%, at least 97%, at least 98%, at least 99%, or 100% sequence identity to SEQ ID NO: 13, wherein the endogenous FAD3C gene encodes a polypeptide having at least 90%, at least 95%, at least 96%, at least 97%, at least 98%, at least 99%, or 100% sequence identity to SEQ ID NO: 14, wherein the endogenous FAD3B gene encodes a polypeptide having at least 90%, at least 95%, at least 96%, at least 97%, at least 98%, at least 99%, or 100% sequence identity to SEQ ID NO: 15, wherein the endogenous Lox1 gene encodes a polypeptide having at least 90%, at least 95%, at least 96%, at least 97%, at least 98%, at least 99%, or 100% sequence identity to SEQ ID NO: 16, wherein the endogenous Lox2 gene encodes a polypeptide having at least 90%, at least 95%, at least 96%, at least 97%, at least 98%, at least 99%, or 100% sequence identity to SEQ ID NO: 17, or wherein the endogenous Lox3 gene encodes a polypeptide having at least 90%, at least 95%, at least 96%, at least 97%, at least 98%, at least 99%, or 100% sequence identity to SEQ ID NO: 18, wherein the endogenous RS2 gene encodes a polypeptide having at least 90%, at least 95%, at least 96%, at least 97%, at least 98%, at least 99%, or 100% sequence identity to SEQ ID NO: 19, or wherein the endogenous RS3 gene encodes a polypeptide having at least 90%, at least 95%, at least 96%, at least 97%, at least 98%, at least 99%, or 100% sequence identity to SEQ ID NO: 20.
9. The soybean plant of any one of embodiments 1-8, wherein the mutant allele comprises one or more nucleotide modifications within the coding region, non-coding region, regulatory sequence, or untranslated region of the endogenous FAD2-1A, FAD2-1B, FAD3A, FAD3C, FAD3B, Lox1, Lox2, Lox3, RS2, and/or RS3 gene.
10. The soybean plant of any one of embodiments 1-9, wherein the one or more nucleotide modifications reduce expression of the endogenous FAD2-1A, FAD2-1B, FAD3A, FAD3C, FAD3B, Lox1, Lox2, Lox3, RS2, and/or RS3 gene; reduce transcriptional activity of the polypeptide encoded by the endogenous FAD2-1A, FAD2-1B, FAD3A, FAD3C, FAD3B, Lox1, Lox2, Lox3, RS2, and/or RS3 gene; generate one or more alternative spliced variants of the endogenous FAD2-1A, FAD2-1B, FAD3A, FAD3C, FAD3B, Lox1, Lox2, Lox3, RS2, and/or RS3 gene; introduce a frameshift mutation in one or more exons of the endogenous FAD2-1A, FAD2-1B, FAD3A, FAD3C, FAD3B, Lox1, Lox2, Lox3, RS2, and/or RS3 gene; delete a substantial portion of the endogenous FAD2-4A, FAD2-1B, FAD3A, FAD3C, FAD3B, Lox1, Lox2, Lox3, RS2, and/or RS3 gene; delete a full-length open reading frame of the endogenous FAD2-4A, FAD2-1B, FAD3A, FAD3C, FAD3B, Lox1, Lox2, Lox3, RS2, and/or RS3 gene; repress an enhancer motif present within a regulatory region encoding the endogenous FAD2-4A, FAD2-1B, FAD3A, FAD3C, FAD3B, Lox1, Lox2, Lox3, RS2, and/or RS3 gene; modify one or more nucleotides of a regulatory element operably linked to the endogenous FAD2-4A, FAD2-1B, FAD3A, FAD3C, FAD3B, Lox1, Lox2, Lox3, RS2, and/or RS3 gene; or any combination thereof.
11. The soybean plant of any one of embodiments 1-10, wherein the one or more improved flavor characteristics comprise high oleic acid and/or low linolenic acid relative to a soybean plant without the mutant allele.
12. A seed or an asexual propagate of the soybean plant of any one of embodiments 1-11.
13. A method for improving one or more flavor characteristics of soybean seed, the method comprising: introducing a mutant allele of the endogenous FAD2-1A, FAD2-1B, FAD3A, FAD3C, FAD3B, Lox1, Lox2, Lox3, RS2, and/or RS3 gene in a soybean plant.
14. The method of embodiment 13, wherein the mutant allele is a loss-of-function allele, a null allele, or a hypomorphic allele.
15. The method of embodiment 13 or embodiment 14, wherein the mutant allele is introduced through traditional breeding techniques or gene editing.
16. The method of any one of embodiments 13-15, wherein the endogenous FAD2-1A gene comprises a nucleotide sequence having at least 90%, at least 95%, at least 96%, at least 97%, at least 98%, at least 99%, or 100% sequence identity to SEQ ID NO: 1, wherein the endogenous FAD2-1B gene comprises a nucleotide sequence having at least 90%, at least 95%, at least 96%, at least 97%, at least 98%, at least 99%, or 100% sequence identity to SEQ ID NO: 2, wherein the endogenous FAD3A gene comprises a nucleotide sequence having at least 90%, at least 95%, at least 96%, at least 97%, at least 98%, at least 99%, or 100% sequence identity to SEQ ID NO: 3, wherein the endogenous FAD3C gene comprises a nucleotide sequence having at least 80%, at least 90%, at least 95%, at least 96%, at least 97%, at least 98%, at least 99%, or 100% sequence identity to SEQ ID NO: 4, wherein the endogenous FAD3B gene comprises a nucleotide sequence having at least 90%, at least 95%, at least 96%, at least 97%, at least 98%, at least 99%, or 100% sequence identity to SEQ ID NO: 5, wherein the endogenous Lox1 gene comprises a nucleotide sequence having at least 90%, at least 95%, at least 96%, at least 97%, at least 98%, at least 99%, or 100% sequence identity to SEQ ID NO: 6, wherein the endogenous Lox2 gene comprises a nucleotide sequence having at least 90%, at least 95%, at least 96%, at least 97%, at least 98%, at least 99%, or 100% sequence identity to SEQ ID NO: 7, wherein the endogenous Lox3 gene comprises a nucleotide sequence having at least 90%, at least 95%, at least 96%, at least 97%, at least 98%, at least 99%, or 100% sequence identity to SEQ ID NO: 8, wherein the endogenous RS2 gene comprises a nucleotide sequence having at least 90%, at least 95%, at least 96%, at least 97%, at least 98%, at least 99%, or 100% sequence identity to SEQ ID NO: 9, or wherein the endogenous RS3 gene comprises a nucleotide sequence having at least 90%, at least 95%, at least 96%, at least 97%, at least 98%, at least 99%, or 100% sequence identity to SEQ ID NO: 10.
17. The method of any one of embodiments 13-16, wherein the endogenous FAD2-1A gene encodes a polypeptide having at least 90%, at least 95%, at least 96%, at least 97%, at least 98%, at least 99%, or 100% sequence identity to SEQ ID NO: 11, wherein the endogenous FAD2-1B gene encodes a polypeptide having at least 90%, at least 95%, at least 96%, at least 97%, at least 98%, at least 99%, or 100% sequence identity to SEQ ID NO: 12, wherein the endogenous FAD3A gene encodes a polypeptide having at least 90%, at least 95%, at least 96%, at least 97%, at least 98%, at least 99%, or 100% sequence identity to SEQ ID NO: 13, wherein the endogenous FAD3C gene encodes a polypeptide having at least 90%, at least 95%, at least 96%, at least 97%, at least 98%, at least 99%, or 100% sequence identity to SEQ ID NO: 14, wherein the endogenous FAD3B gene encodes a polypeptide having at least 90%, at least 95%, at least 96%, at least 97%, at least 98%, at least 99%, or 100% sequence identity to SEQ ID NO: 15, wherein the endogenous Lox1 gene encodes a polypeptide having at least 90%, at least 95%, at least 96%, at least 97%, at least 98%, at least 99%, or 100% sequence identity to SEQ ID NO: 16, wherein the endogenous Lox2 gene encodes a polypeptide having at least 90%, at least 95%, at least 96%, at least 97%, at least 98%, at least 99%, or 100% sequence identity to SEQ ID NO: 17, or wherein the endogenous Lox3 gene encodes a polypeptide having at least 90%, at least 95%, at least 96%, at least 97%, at least 98%, at least 99%, or 100% sequence identity to SEQ ID NO: 18, wherein the endogenous RS2 gene encodes a polypeptide having at least 90%, at least 95%, at least 96%, at least 97%, at least 98%, at least 99%, or 100% sequence identity to SEQ ID NO: 19, or wherein the endogenous RS3 gene encodes a polypeptide having at least 90%, at least 95%, at least 96%, at least 97%, at least 98%, at least 99%, or 100% sequence identity to SEQ ID NO: 20.
18. The method of any one of embodiments 13-17, wherein the mutant allele comprises one or more nucleotide modifications within the coding region, non-coding region, regulatory sequence, or untranslated region of the endogenous FAD2-1A, FAD2-1B, FAD3A, FAD3C, FAD3B, Lox1, Lox2, Lox3, RS2, and/or RS3 gene.
19. The method of any one of embodiments 13-18, wherein the one or more nucleotide modifications reduce expression of the endogenous FAD2-1A, FAD2-1B, FAD3A, FAD3C, FAD3B, Lox1, Lox2, Lox3, RS2, and/or RS3 gene; reduce transcriptional activity of the polypeptide encoded by the endogenous FAD2-1A, FAD2-1B, FAD3A, FAD3C, FAD3B, Lox1, Lox2, Lox3, RS2, and/or RS3 gene; generate one or more alternative spliced variants of the endogenous FAD2-1A, FAD2-1B, FAD3A, FAD3C, FAD3B, Lox1, Lox2, Lox3, RS2, and/or RS3 gene; introduce a frameshift mutation in one or more exons of the endogenous FAD2-4A, FAD2-1B, FAD3A, FAD3C, FAD3B, Lox1, Lox2, Lox3, RS2, and/or RS3 gene; delete a substantial portion of the endogenous FAD2-4A, FAD2-1B, FAD3A, FAD3C, FAD3B, Lox1, Lox2, Lox3, RS2, and/or RS3 gene; delete a full-length open reading frame of the endogenous FAD2-1A, FAD2-1B, FAD3A, FAD3C, FAD3B, Lox1, Lox2, Lox3, RS2, and/or RS3 gene; repress an enhancer motif present within a regulatory region encoding the endogenous FAD2-1A, FAD2-1B, FAD3A, FAD3C, FAD3B, Lox1, Lox2, Lox3, RS2, and/or RS3 gene; modify one or more nucleotides of a regulatory element operably linked to the endogenous FAD2-1A, FAD2-1B, FAD3A, FAD3C, FAD3B, Lox1, Lox2, Lox3, RS2, and/or RS3 gene; or any combination thereof.
20. The method of any one of embodiments 13-19, wherein the one or more improved flavor characteristics comprise high oleic acid and/or low linolenic acid relative to a soybean plant without the mutant allele.
21. A commodity plant product prepared from the soybean plant, plant part, or plant cell of any one of embodiments 1-11.
22. The commodity plant product of embodiment 21, wherein the product is protein, white flakes, flakes, seed meal, fodder, oil, soymilk, or seed-treatment-coated seed.
23. A method for producing a commodity plant product, the method comprising processing the soybean plant or plant part of any one of embodiments 1-11 to obtain the product.
24. The method of embodiment 23, wherein the product is protein, white flakes, flakes, seed meal, fodder, oil, soymilk, or seed-treatment-coated seed.
25. A soybean crop comprising a plurality of the soybean plants of any one of embodiments 1-11 planted together in an agricultural field.
26. A method of producing seeds from a soybean crop, the method comprising: cultivating a plurality of the soybean plants of any one of embodiments 1-11 as a soybean crop, and harvesting seeds from the soybean crop.
27. A method of producing a soybean plant having one or more improved flavor characteristics, the method comprising: (a) crossing the soybean plant of any one of embodiments 1-11 with itself or another soybean plant to produce a seed; and (b) growing a progeny soybean plant from the seed to produce a soybean plant having one or more improved flavor characteristics.
28. The method of embodiment 27, further comprising: (c) crossing the progeny soybean plant with itself or another soybean plant; and (d) repeating steps (b) and (c) for an additional 0-7 generations to produce a progeny soybean plant of a subsequent generation having one or more improved flavor characteristics.
29. A method of introgressing a locus associated with one or more improved flavor characteristics into a soybean plant, the method comprising: providing a first soybean plant with the locus associated with one or more improved flavor characteristics, wherein the locus comprises a mutant allele of the endogenous FAD2-1A, FAD2-1B, FAD3A, FAD3C, FAD3B, Lox1, Lox2, Lox3, RS2, and/or RS3 gene; providing a second soybean plant; crossing the first soybean plant with the second soybean plant to produce a population of progeny soybean plants; selecting from the population at least one soybean plant having the locus associated with one or more improved flavor characteristics.
30. The method of embodiment 29, further comprising performing backcrosses and selections.
31. The method of embodiment 29 or embodiment 30, wherein the introgressing comprises marker assisted selection.
32. The method of any one of embodiments 29-31, wherein the second soybean plant comprises a mutant allele of the endogenous FAD2-1A, FAD2-1B, FAD3A, FAD3C, FAD3B, Lox1, Lox2, Lox3, RS2, and/or RS3 gene.

### EXAMPLES

### Example 1: Soybean Germplasm

Soybean seeds represent an underutilized stock as an ingredient source or component of manufactured products for nutritious protein-rich human foods. Currently, undesirable flavors from soybean-derived ingredients prevent the full inclusion of soy products in foods. This example describes a genetic combination of soybean traits (mutations in seven to ten identified genes) for improved soybean seed composition with reduced off-flavors by changing the chemical makeup of the seed. Eight of the mutant genes change the fatty acid profile of the seed oil and the seed lipoxygenase enzyme activity to reduce off-flavors from lipid oxidation during processing, while two of the mutant genes alter the seed carbohydrate profile that increases sucrose and nearly eliminates the undesirable raffinose family of oligosaccharides. The mutant gene combination results in soybean seeds with very low levels of polyunsaturated fatty acids (PUFAs) in the seed oil and also disables the lipoxygenase enzymes that attack the PUFAs to create the off-flavor chemicals. The Super-L soybean germplasm designation represents mutations in the ten genes which were identified and combined into one germplasm line as a composition bundle; similarly, mutations in eight genes can be combined into a composition bundle designated here as HOLL+LIPOX (Table 1). The combination of nine mutant genes (all except the mutant FAD3B gene) is referred to as Super (Table 2). Table 3 summarizes the mutant alleles present in the lines. These seed composition changes reduce or eliminate production of the lipid oxidation-related chemicals responsible for many of the soy off-flavors. Soybean seeds and processed products made from the seeds with the gene mutation combinations have improved flavor profiles and can therefore be utilized more readily in foods.

TABLE 1

| Type | | Trait | Component | Gene | Wm82.a2.v1 |
|---|---|---|---|---|---|
| Super-L | HOLL + LIPOX | HOLL | Fatty acid profile | FAD2-1A | Glyma.10g278000 |
| | | | | FAD2-1B | Glyma.20g111000 |
| | | | | FAD3A | Glyma.14g194300 |
| | | | | FAD3C | Glyma.18g062000 |
| | | | | FAD3B | Glyma.02g227200 |
| | | LIPOX | Lipoxygenase | Lox1 | Glyma.13g347600 |
| | | | enzyme activity | Lox2 | Glyma.13g347500 |
| | | | | Lox3 | Glyma.15g026300 |
| | | CARB | Carbohydrate | RS2 | Glyma.06g179200 |
| | | | profile | RS3 | Glyma.05g003900 |

TABLE 2

| Type | Component | Gene | Wm82.a2.v1 |
|---|---|---|---|
| Super | Carbohydrate | RS2 | Glyma.06g179200 |
| | profile | RS3 | Glyma.05g003900 |
| | Fatty acid profile | FAD2-1A | Glyma.10g278000 |
| | | FAD2-1B | Glyma.20g111000 |
| | | FAD3A | Glyma.14g194300 |
| | | FAD3C | Glyma.18g062000 |
| | Lipoxygenase | Lox1 | Glyma.13g347600 |
| | | Lox2 | Glyma.13g347500 |
| | | Lox3 | Glyma.15g026300 |

TABLE 3

| | | |
|---|---|---|
| Gene | Wm82.a2.v1 | Mutant allele |

| | | | |
|---|---|---|---|
| FAD2-1A | Glyma.10g278000 | InDel | frameshift |
| FAD2-1B | Glyma.20g111000 | P137R | Missense |
| FAD3A | Glyma.14g194300 | Splice | Null-splice |
| FAD3C | Glyma.18g062000 | G128E | Missense |
| FAD3B | Glyma.02g227200 | splice | Null-splice |
| RS2 | Glyma.06g179200 | rs2W331- | frameshift |
| RS3 | Glyma.05g003900 | rs3G75E | Missense |
| Lox1 | Glyma.13g347600 | InDel | frameshift |
| Lox2 | Glyma.13g347500 | H532Q | Missense |
| Lox3 | Glyma.15g026300 | InDel | frameshift |

### Example 2: Preliminary Seed Fatty Acid Analysis

A preliminary analysis of fatty acids in seed oil was conducted for the Super-L soybean germplasm compared to other mutant gene combinations (Table 4). The Super soybean type produced a typical high oleic and low linolenic acid (HOLL) seed oil profile that met informal industry targets of >75% oleic acid and less than 3% linolenic acid. The Super-L seed samples had reduced linolenic acid in the seed oil and overall, the lowest PUFAs in soybean seeds that have been generated to date.

TABLE 4

| | % fatty acid in oil | | | | |
|---|---|---|---|---|---|
| | palmitic | stearic | oleic | linoleic | linolenic |
| | 16:0 | 18:0 | 18:1 | 18:2 | 18:3 |
| Patriot | 11.7 | 4.4 | 23.8 | 53.2 | 6.9 |
| HOLL + LIPOX | 7.2 | 3.1 | 83.9 | 3.5 | 2.3 |
| Super | 7.5 | 3.3 | 83.9 | 3.2 | 2.2 |
| Super-L | 8.0 | 3.8 | 83.9 | 3.5 | 0.9 |

### Example 3: Sensory Attributes

Soybean samples (ground seed slurries) were analyzed for sensory attributes by a trained panel. This analysis did not include Super-L samples because appropriate quantities of seed were not yet available. The commodity soybean was the only sample characterized as painty. The Super soybean (nine genes) and HOLL+LIPOX (7 genes) had sensory profiles that included low scores for negative flavor attributes (Table 5). Together, this information led to the development of soybean germplasm with the eight or ten mutant gene combination that further reduces linolenic acid levels in the seed oil to create a soybean type with no off-flavors for expanded use in ingredients and foods.

TABLE 5

| | | | cereal / | | | | | | | |
|---|---|---|---|---|---|---|---|---|---|---|
| | aroma | sweet | grain / | | nutty / | | | | astr. | after |
| | intensity | aromatic | flour | beany | roasted | sweet | bitter | umami | mf | taste |
| Commodity | 3.9a | 1.0b | ND | 1.1f | ND | 2.1ab | 1.0a | 2.2c | 3.3a | 3.3a |
| HOLL | 3.1b | 0.5c | 1.0c | 2.0e | 1.0d | 1.6c | ND | 1.9d | 2.8bc | 2.1c |
| TIGER | 3.1b | 2.4a | 0.6d | 3.4a | 0.6e | 2.2a | ND | 3.6a | 2.6c | 2.1c |
| SUPER | 3.1b | 1.1b | 1.3b | 2.8c | 1.0c | 2.0b | ND | 2.6b | 2.5c | 2.2c |
| HOLL + | 2.1c | 1.1b | 1.2bc | 3.1b | 0.8de | 2.0b | ND | 2.0cd | 3.0b | 1.9d |
| LIPOX | | | | | | | | | | |
| Bl Super | 3.2b | 2.4a | 2.2a | 3.0b | 2.0a | 2.0b | 0.7b | 2.4b | 3.0b | 2.5b |
| aromatic | | | | | | | | | | |
| Y Super | 3.0b | 2.5a | 2.0a | 2.6d | 1.3b | 2.1ab | ND | 3.4a | 2.5c | 2.6b |
| aromatic | | | | | | | | | | |

### Example 4: Sensory and Instrumental Analysis of Soybean and Almond Slurries

This example shows the seed composition changes of the soybean germplasm using Super as a baseline to investigate further flavor improvements (Table 6). A blanched almond control as well as the commodity Patriot soybean control were also included. Table 7 shows the fatty acid profile of the seed oil, and Table 8 shows seed sucrose, nitrogen, protein, and oil content.

TABLE 6

| | | | | | | | Super- | HOLL + |
|---|---|---|---|---|---|---|---|---|
| | Patriot | MUT656 | MUT1792 | MUT2401 | Super | Super-L | aro | LIPOX |
| FAD2-1A | functional | mutant | mutant | mutant | mutant | mutant | mutant | mutant |
| FAD2-1B | functional | mutant | mutant | mutant | mutant | mutant | mutant | mutant |
| FAD3A | functional | mutant | mutant | mutant | mutant | mutant | mutant | mutant |
| FAD3C | functional | mutant | mutant | mutant | mutant | mutant | mutant | mutant |
| RS2 | functional | mutant | mutant | mutant | mutant | mutant | mutant | functional |
| RS3 | functional | mutant | mutant | mutant | mutant | mutant | mutant | functional |
| Lox1 | functional | mutant | mutant | mutant | mutant | mutant | mutant | mutant |
| Lox2 | functional | mutant | mutant | mutant | mutant | mutant | mutant | mutant |
| Lox3 | functional | mutant | mutant | mutant | mutant | mutant | mutant | mutant |
| FAD3B | functional | functional | functional | functional | functional | mutant | functional | functional |
| Bad2 | functional | functional | functional | functional | functional | functional | mutant | functional |

TABLE 7

| | palmitic | stearic | oleic | linoleic | linolenic |
|---|---|---|---|---|---|
| Patriot | 11.3 | 4.4 | 23.7 | 53.4 | 7.3 |
| MUT656 | 7.4 | 4.0 | 83.3 | 3.3 | 2.1 |
| MUT1792 | 7.5 | 3.5 | 83.5 | 3.3 | 2.2 |
| MUT2401 | 7.7 | 3.6 | 82.5 | 4.0 | 2.2 |
| Super | 7.7 | 4.1 | 81.3 | 4.3 | 2.5 |
| Super-L | 7.8 | 3.9 | 84.0 | 3.5 | 0.8 |
| Super-aro | 7.5 | 3.7 | 83.3 | 3.2 | 2.1 |
| HOLL + LIPOX | 7.0 | 3.2 | 84.4 | 3.4 | 2.1 |

TABLE 8

| | | | Predicted | |
|---|---|---|---|---|
| | | | Protein | Predicted |
| | Seed sucrose | % seed | Dry basis | Oil Dry |
| | (% dw) | protein | % | basis % |
| Patriot | 5.8 | 33.8 | 39.4 | 20.9 |
| MUT656 | 7.2 | 36.3 | 38.7 | 23.3 |
| MUT1792 | 7.1 | 39.2 | 42.3 | 20.4 |
| MUT2401 | 6.1 | 36.1 | 40.7 | 21.8 |
| Super | 6.7 | 39.9 | 39.0 | 21.9 |
| Super-L | 7.9 | 37.4 | 40.4 | 20.4 |
| Super-aro | 7.5 | 40.2 | 42.0 | 21.2 |
| HOLL + LIPOX | 5.6 | 34.3 | 37.1 | 23.0 |

### Descriptive Analysis

Preparations were conducted with the overhead lights off to prevent light-induced off-flavor formation. Following preparation, products were dispensed into lidded soufflé cups with 3-digit codes and evaluated at 15° C. Slurries were evaluated in duplicate by 6 trained panelists using an established sensory language for plant proteins the day following sample preparation. Descriptive analysis of flavor used a 0 to 15 point universal intensity scale with the Spectrum method. Paper ballots were used. Each panelist evaluated each product in duplicate in different sessions. Table 9 shows the sensory attributes of rehydrated soy and almond slurries.

TABLE 9

| | | | | | | | Super- | HOLL + | |
|---|---|---|---|---|---|---|---|---|---|
| | Patriot | MUT656 | MUT1792 | MUT2401 | Super | Super-L | aro | LIPOX | Almond |
| aroma | 4.2a | 2.5c | 2.7c | 2.8c | 3.2b | 3.0b | 3.2b | 3.7ab | 1.6d |
| intensity | | | | | | | | | |
| overall | 5.1a | 4.2c | 3.3e | 3.7d | 3.1f | 4.6b | 4.6b | 4.5b | 4.2c |
| flavor | | | | | | | | | |
| impact | | | | | | | | | |
| sweet | ND | 0.6d | 1.4b | 0.8d | 1.4b | 1.1c | 0.6 | ND | 2.3a |
| aromatic | | | | | | | | | |
| card - | 3.2a | 1.6c | ND | ND | ND | ND | 1.3d | 1.9b | ND |
| board | | | | | | | | | |
| green | ND | 2.4a | 0.9d | 1.3b | 0.8d | 0.9d | 1.1c | 1.1c | ND |
| painty | 4.6a | 1.1b | ND | ND | ND | ND | ND | ND | ND |
| beany | 2.1b | 2.3b | 2.1b | 2.6a | 1.4c | 2.6a | 2.6a | 2.7a | ND |
| brothy / | ND | 1 | ND | ND | ND | ND | ND | ND | ND |
| serumy | | | | | | | | | |
| sweet | 2.0a | 1.7b | 1.9a | 1.7b | 2.0a | 2.0a | 1.7b | 1.7b | 1.6b |
| bitter | 1.0b | 0.5c | 0.5c | 1.3b | 0.8bc | 1.1b | 1.3b | 2.0a | 0.5 |
| umami | 3.3a | 3.2a | 3.1a | 3.0a | 2.7b | 2.7b | 3.2a | 2.5 | 1.2c |
| astr. mf | 3.4ab | 2.7c | 3.1b | 3.6a | 3.5a | 3.1b | 3.1b | 3.8a | 2.3d |
| after | 3.1a | 2.8b | 2.7b | 2.7b | 2.6b | 2.3c | 2.2c | 2.4bc | 1.4d |
| taste | | | | | | | | | |

Sensory attributes are scored on a 0 to 15 point universal intensity scale.
Most dried dairy ingredient attributes fall between 0 and 4.
ND—not detected.

### Instrumental Volatile Compound Analysis

All injections were made on an Agilent 7820 GC with 5975 MSD with a DB-5 ms (30 m×0.25 mm ID×0.25 am) column. Sample introduction was accomplished using a CTC Analytics CombiPal Autosampler. Five (5) g of soy slurry was added to 20 ml SPME vials. Vials were equilibrated for 25 min at 40° C. with 4 second pulsed 250 rpm agitation. 20 μl of internal standard (2-methyl-3-heptanone in methanol at 10 ppm) was added to each vial. A single DVB/Carboxen/PDMS 1 cm fiber was used for all analysis. The SPME fiber was exposed to the samples for 40 min at depth 3.1 cm. The fiber was retracted and injected at 5.0 cm in the GC inlet for 5 minutes. SCAN method was performed to identify compound of interest. Samples were analyzed in triplicate.

GC conditions oven program: 40° C. for 3 minutes; 10° C./min to 90° C.; 5° C./min to 200° C.; hold 10 minute; 20° C./min to 250° C.; hold 5 minutes; injector at 250° C. splitless; constant flow rate of 1 ml/min. MSD conditions: source 250 NC; quad 150° C.; MSD transfer line 250.

Table 10 shows the relative mean concentration of selected volatile compounds in rehydrated soy and almond slurries (ppb).

TABLE 10

| | | | Super- | | | | HOLL + | Super- | |
|---|---|---|---|---|---|---|---|---|---|
| Compound | Patriot | MUT656 | aro | MUT1792 | Super | MUT2401 | LIPOX | L | Almond |
| acetic acid | 4.09 a | 4.26 a | ND | ND | ND | ND | ND | ND | ND |
| methyl ester | ND | ND | ND | ND | ND | ND | ND | ND | ND |
| acetic acid | | | | | | | | | |
| pentyl ester | ND | ND | ND | ND | ND | ND | ND | ND | ND |
| acetic acid | | | | | | | | | |
| a-pinene | ND | ND | ND | ND | ND | ND | ND | 0.73 b | 16 a |
| benzaldehyde | 7.93 b | ND | 9.88 b | 21.44 b | 9.76 b | 4.81 b | 9.30 b | 3.91 b | 194.59 a |
| butanal | 4.35 a | 2.83 cd | 3.46 bc | ND | ND | ND | 4.24 ab | 2.32 d | ND |
| (E,E)-2,4- | 1.08 a | ND | ND | ND | ND | ND | ND | ND | ND |
| decadienal | | | | | | | | | |
| Dimethyl | ND | 5.27 b | 42.41 a | 39.87 a | 38.51 a | 1.56 b | 26.14 ab | 21.22 ab | ND |
| sulfide | | | | | | | | | |
| carbon | ND | 9.72 c | 34.95 b | 37.52 b | ND | 10.39 c | 80.28 a | ND | ND |
| disulfide | | | | | | | | | |
| 2-ethylfuran | 42 a | ND | 3.09 b | 2.38 bc | ND | 4.01 b | 1.88 bc | ND | ND |
| 2-pentylfuran | 72.43 a | 8.54 b | ND | 2.66 cd | ND | 7.29 bc | ND | 4.59 bcd | 3.41 bcd |
| 1-heptanol | ND | ND | ND | ND | ND | ND | ND | ND | ND |
| 2-heptanone | 6.92 a | ND | ND | ND | ND | ND | ND | ND | ND |
| heptanal | 7.20 a | 6.80 a | ND | ND | ND | ND | ND | ND | ND |
| (E)-2 | ND | ND | ND | ND | ND | ND | ND | ND | ND |
| heptenal | | | | | | | | | |
| hexanal | 1265.62 a | 660.01 b | 522.06 cd | 436.92 de | 596.43 bc | 510.64 cd | 382.03 ef | 286.98 f | 13.98 g |
| (E)-2- | 2.60 e | 3.74 de | 6.24 abc | 5.85 bc | 8.05 a | 6.99 ab | 5.06 cd | 2.45 e | ND |
| hexenal | | | | | | | | | |
| 1-hexanol | 47.17 a | 8.98 c | 9.49 c | 9.91 c | 8.49 c | 12.88 c | 3.52 d | 21.88 b | 3.01 d |
| methyl ester | ND | ND | ND | ND | ND | 1.66 a | ND | ND | ND |
| hexanoic | | | | | | | | | |
| acid | | | | | | | | | |
| 2-methyl-1- | ND | 2.88 b | ND | 1.98 b | 2.28 b | ND | ND | 4.86 a | 5.21 a |
| butanol | | | | | | | | | |
| 2-methyl- | 1.94 d | 3.04 cd | 5.52 b | 5.79 b | 8.03 a | 3.21 cd | 5.98 b | 3.58 c | 5.33 b |
| furan | | | | | | | | | |
| 2-nonenal | ND | ND | ND | ND | ND | ND | ND | ND | ND |
| (E,E)-2,4- | ND | ND | ND | ND | ND | ND | ND | ND | ND |
| nonadienal | | | | | | | | | |
| nonanal | 2.06 d | 12.55 a | 5.42 b | 3.86 c | 4.63 bc | 3.51 c | 3.42 c | 3.41 c | ND |
| octanal | 0.94 b | 2.38 a | 2.10 a | ND | ND | 0.86 b | ND | 1.12 b | ND |
| 2,3- | 13.26 a | 5.73 b | ND | ND | ND | ND | ND | ND | ND |
| octanedione | | | | | | | | | |
| 2-octen-1-ol | ND | ND | ND | ND | ND | ND | ND | ND | ND |
| (E)-2-octenal | 2.04 a | ND | ND | ND | ND | ND | ND | ND | ND |
| 1-octanol | ND | ND | ND | ND | ND | ND | ND | ND | ND |
| 1-octen-3-ol | 12.67 cd | 45.97 b | 17.9 c | 8.19 d | 46.59 b | 10.83 cd | 75.1 a | 14.68 cd | ND |
| 3-octen-2- | 3.55 a | ND | ND | ND | ND | ND | ND | ND | ND |
| one | | | | | | | | | |
| 2-penten-1-ol | ND | ND | ND | ND | ND | ND | ND | ND | ND |
| (E)-2- | 2.55 a | ND | ND | ND | ND | ND | ND | ND | ND |
| pentenal | | | | | | | | | |
| 1-pentanol | 42.79 a | 9.37 b | ND | ND | ND | ND | ND | ND | ND |
| 1-penten-3-ol | 241.11 a | 65.03 b | 6.86 c | 7.52 c | 7.55 c | 12.22 c | 4.98 c | 3.80 c | ND |

ND: Not detected
Mean values in triplicate
Means followed by different letters in rows are significantly different (p < 0.05)

### Example 5: Volatile Profiles of Soymilk Made from Aged Soybeans

Four soybean lines grown in 2022 in Columbia, MO included Patriot (commodity, Patriot) and three experimental lines KB 18-15 #1515 (HOLL), KB18-35 #1809/1810 (HOLL/ULRFO, Tiger), and KB 19-7 #1328 (HOLL/ULRFO/LJPOX, Super). The germplasm lines were developed to enhance the value of specialty soybeans through seed composition improvement. The combinations of variant alleles of structural genes were selected for controlling the fatty acid profile of the oil (HOLL; FAD2-1A, FAD2-1B, FAD3A, and FAD3C), the carbohydrate profile (ULRFO; rs2 and rs3), and lipoxygenase enzymes (LIPOX; lox1, lox2, and lox3).

### Preparation of Aged Soybean

Whole dry beans were weighed (50 g) and placed in an 80 ml beaker. Samples were incubated in a Fischer Scientific Isotemp Incubator 650D for 10 days at 50° C.

### Preparation of Soymilk

Whole dry beans were weighed 50 g then cleaned using deionized (DI) water. The beans were placed in a beaker with DI water at 1:5 w/w ratio. The beans were left at room temperature to soak for 16 hours. The soaking water was discarded. The beans were ground with fresh DI (1:10 beans to water ratio) using a THERMOMIX® TM5 (Vorwerk & Co. KG, Wuppertal, Germany). Grinding occurred for 6 minutes at speed 9 (7600 rpm). No heat was applied. Following grinding, the soy slurry was heated at 100° C. for 12 minutes and stirred at speed 5 (2000 rpm) to produce soymilk. The sample was weighed and adjusted using DI water to 500 g to account for evaporation during heating. Overall, soymilk formulations consisted of 44 g of soybeans in 456 g of DI water. No additional sugar, stabilizer, or emulsifier was added.

### HS-SPME-GC-MS/MS Analysis

Volatile analysis was conducted using the Headspace Solid-Phase Microextraction Gas Chromatography Mass Spectrometry Analysis (HS-SPME-GC-MS/MS) method.

The compounds represented by the features were tentatively identified using the NIST MS Search v2.2, NIST 14 Mass Spectral Library database (Scientific Instrument Services, Ringoes, NJ, USA) by matching the mass spectral data with that of the compound. Only the compounds that had a high match score (over 700) to the NIST database were considered. Additionally, linear retention indices (RI) were calculated using Kovats' equation from a sequence of linear hydrocarbons from C₇ to C₃₀ to verify the NIST match with that of literature. Thus, two-step identification was made for the volatile compounds as possible matches were identified by comparison of the mass spectral data within the NIST library and then verified as a valid prospect based on RI data. Odor thresholds of compounds found in the literature were used to calculate their odor activity values which show the relative contribution of each volatile compound to the final odor of the soymilk. Odor activity values were calculated using the equation provided below:

$$Odor activity = \frac{Concentration of Compound}{Concentration of odor threshold} .$$

### Results

Analysis identified numerous compounds, with traditional commodity soybeans (Patriot) showing the highest concentrations of off-flavor compounds like hexanal, 2-pentylfuran, and 2-ethylfuran-all associated with undesirable beany flavors. HOLL and Tiger varieties exhibited fewer volatile compounds due to their reduced linoleic and linolenic acid content, which limited substrate availability for oxidation. Super soybeans, which combine modified fatty acid profiles with lipoxygenase-null traits, demonstrated the most significant improvement, containing just three compounds with odor activity values above threshold (nonanal, octanal, and hexanal), with hexanal concentrations 230 times lower than in Patriot. The removal of lipoxygenase enzymes proved particularly effective in reducing beany off-flavors, while modified carbohydrate composition (reduced raffinose and stachyose) appeared to have minimal impact on flavor development. These findings suggest that combining fatty acid modifications with lipoxygenase elimination offers the most promising approach for producing soymilk with significantly improved flavor profiles. Table 11 shows the characteristics for major volatile compounds identified in aged soymilk. Table 12 shows the major volatile compounds in aged soymilk.

TABLE 11

| Compound | OT^{b} | Odor description |
|---|---|---|
| Aldehydes (8) | | |
| Hexanal | 4.5 | Green, beany |
| Heptanal | 3 | Fat, rancid, citrus |
| Octanal | 0.70 | Fat, soap, green |
| Nonanal | 1 | Fat, citrus, green |
| (E)-2-Heptenal | 13 | Soap, fat |
| (E)-2-Hexenal | 190 | Leafy |
| (E, E)-2,4- | 10 | Spices |
| Decadienal | | |
| Benzaldehyde | 350 | Almond, bitter |
| Alcohols (2) | | |
| 2-Penten-1-ol | 89.2 | Green |
| 2-Hexanol | 1508.2 | Fruity, fat |
| Ketones (2) | | |
| 3-Octen-2-one | 6.7 | Mushroom, green |
| 2-Heptanone | 3000 | Fragrance |
| Acids (3) | | |
| Acetic acid | 180,000 | Sour |
| Hexanoic acid | 3000 | Sweaty |
| Pentanoic acid | 10000 | Green, cheesy |
| Furan (3) | | |
| 2-Ethyl furan | 2.3 | Beany, green, Sweet |
| 2-Pentyl furan | 5 | Beany |
| 2-n-butyl furan | 6 | Weak |

^{a} RI: Retention indices;
^{b}OT: Odor thresholds in water (ppb).
ND: not detected.

TABLE 12

| | Relative concentration (μg/kg)* | | | | OAV** | | | |
|---|---|---|---|---|---|---|---|---|
| Compound | Patriot | HOLL | Tiger | Super | Patriot | HOLL | Tiger | Super |
| Aldehydes (8) | | | | | | | | |
| Hexanal | 3043^{a} | 344^{b} | 192^{b} | 13.2^{b} | 676 | 76.4 | 42.6 | 2.93 |
| Heptanal | 58.0^{a} | 67.3^{a} | 20.3^{b} | ND | 19.3 | 22.4 | 6.75 | |
| Octanal | ND | 57.2^{a} | 10.1^{b} | 6.01^{b} | — | 81.7 | 14.5 | 8.59 |
| Nonanal | 91.3^{a} | 73.2^{a} | 25.2^{b} | 10.4^{b} | 91.3 | 73.2 | 25.2 | 10.4 |
| (E)-2-Heptenal | 413 | ND | ND | ND | 31.8 | | | |
| (E)-2-Hexenal | 245^{a} | ND | 16.4^{b} | ND | 1.29 | | 0.09 | |
| (E, E)-2,4- | 255 | ND | ND | ND | 25.5 | | | |
| Decadienal | | | | | | | | |
| Benzaldehyde | 70.4^{a} | 34.0^{b} | 27.3^{b} | 19.3^{b} | 0.20 | 0.10 | 0.08 | 0.06 |
| Alcohols (2) | | | | | | | | |
| 2-Penten-1-ol | 60.0 | ND | ND | ND | 0.67 | | | |
| 2-Hexanol | 179^{a} | 236^{a} | 188^{a} | 234^{b} | 0.12 | 0.16 | 0.12 | 0.16 |
| Ketones (2) | | | | | | | | |
| 3-Octen-2-one | 180 | ND | ND | ND | 26.9 | | | |
| 2-Heptanone | 221^{a} | 11.4^{b} | ND | ND | 0.07 | 0.00 | | |
| Acid (3) | | | | | | | | |
| Acetic acid | ND | 27.2^{b} | 44.4^{a} | ND | | 0.00 | 0.00 | |
| Hexanoic acid | 44.2 | ND | ND | ND | 0.01 | | | |
| Pentanoic acid | 560^{a} | 21.3^{b} | ND | 4.06^{b} | 0.06 | 0.00 | | 0.00 |
| Furans (3) | | | | | | | | |
| 2-Ethyl furan | 419^{a} | 73.1^{b} | 43.3^{b} | ND | 182 | 31.8 | 18.8 | |
| 2-Pentyl furan | 3799^{a} | 126^{b} | 31.1^{b} | ND | 633 | 21.0 | 5.19 | |
| 2-n-butyl furan | 69.2 | ND | ND | ND | 13.8 | | | |

*Data represent means ± standard errors.
Different letters denote significant difference between samples within columns (p < 0.05).
ND: not detected.
**OAVs were shown as mean values.

### REFERENCES

Feng, X.; Hua, Y. The precursors of C8 alcohols from soybean: Purification, distribution and hydrolysis properties of glycosidically bound volatiles. Journal of Food Science 2022, 87 (1), 178-192.
Gini, A.; Osako, K.; Ohshima, T. Identification and characterisation of headspace volatiles of fish miso, a Japanese fish meat based fermented paste, with special emphasis on effect of fish species and meat washing. Food Chemistry 2010, 120 (2), 621-631.
Kim, J.; Scaboo, A.; Rainey, K. M.; Fritschi, F. B.; Bilyeu, K. Redesigning soybean with improved oil and meal traits. Theoretical and Applied Genetics 2024, 137 (10), 218.
Lenis, J. M.; Gillman, J. D.; Lee, J. D.; Shannon, J. G.; Bilyeu, K. D. Soybean seed lipoxygenase genes: molecular characterization and development of molecular marker assays. TheorAppl Genet 2010, 120 (6), 1139-1149.
Nedele, A.-K.; Schiebelbein, R.; Bar, A.; Kaup, A.; Zhang, Y. Reduction of aldehydes with green odor in soy products during fermentation with Lycoperdon pyriforme and analysis of their degradation products. Food Research International 2022, 152, 110909.
Pham, A. T.; Shannon, J. G.; Bilyeu, K. D. Combinations of mutant FAD2 and FAD3 genes to produce high oleic acid and low linolenic acid soybean oil. Theor Appl Genet 2012, 125 (3), 503-515.
Stephan, A.; Steinhart, H. Quantification and sensory studies of character impact odorants of different soybean lecithins. Journal of Agricultural and Food Chemistry 1999, 47 (10), 4357-4364.
Varlet, V.; Prost, C.; Serot, T. Volatile aldehydes in smoked fish: Analysis methods, occurrence and mechanisms of formation. Food Chemistry 2007, 105 (4), 1536-1556.
Wang, B.; Zhang, Q.; Zhang, N.; Bak, K. H.; Soladoye, O. P.; Aluko, R. E.; Fu, Y.; Zhang, Y. Insights into formation, detection and removal of the beany flavor in soybean protein. Trends in Food Science & Technology 2021, 112, 336-347.
Yang, D. S.; Lee, K. S.; Jeong, O. Y.; Kim, K. J.; Kays, S. J. Characterization of volatile aroma compounds in cooked black rice. Journal of Agricultural and Food Chemistry 2008, 56 (1), 235-240.

### Example 6: Improved Volatile Profiles and Enhanced Storage Stability of Soy Protein Isolate Through the Development of Novel Soybeans

Soybean lines with improved traits such as high oleic and low linolenic acid (HOLL), ultra-low raffinose and stachyose (ULRFO), and absence of lipoxygenase (LIPOX) have been developed to address the challenge of off-flavors in soy protein isolate (SPI), a major protein ingredient in foods. This example aimed to determine the effects of different soybean seed composition traits on the volatile profile and storage stability of SPIs.

Four soybean lines were investigated: Commodity (Patriot), HOLL, HOLL/ULRFO (Tiger), and HOLL/ULRFO/LIPOX (Super). SPIs were prepared from cold press meals using alkaline extraction, acid precipitation, and freeze-drying. Volatile profiles were determined by headspace solid-phase microextraction combined with gas chromatography-mass spectrometry at time 0 and after 3-month storage at 50° C. and 30% or 75% relative humidity (RH). Data were evaluated by ANOVA with means separation.

Soybean line significantly influenced the types and relative concentrations of volatile compounds in SPIs. Patriot exhibited higher concentrations of total volatiles, aldehydes, and alcohols (p<0.05), while Super showed the lowest concentrations, even lower than HOLL and Tiger (p<0.05). Major off-flavor compounds such as hexanal, 1-hexanol, 2-pentylfuran, and 1-octen-3-ol were significantly higher in Patriot (p<0.05), while Super contained the lowest concentrations of hexanal and heptanal (p<0.05). After storage, Patriot continued to have higher concentrations of total volatiles, aldehydes, and alcohols (p<0.05), while Super exhibited the lowest concentrations. Larger changes in volatile contents were observed at 30% RH compared to 75% RH, with Super exhibiting the smallest changes, particularly at 75% RH. In conclusion, HOLL, Tiger, and Super showed lower off-flavors compared to Patriot, with Super producing SPI with the lowest concentrations of off-flavor compounds and better storage stability, demonstrating its potential as a protein ingredient with higher consumer acceptability. PCA analysis revealed distinctive volatile profiles for Patriot and Super, while HOLL and Tiger displayed more similar characteristics. Correlation analysis confirmed Super SPI's association with significantly fewer volatile compounds, demonstrating its superior flavor profile and enhanced oxidative stability-making it the most promising candidate for improved soy protein applications.

Table 13 shows the relative contents of volatile compounds in freshly made SPIs, and Table 14 shows the relative OAVs and odor description of major volatile compounds in freshly made SPIs.

TABLE 13

| | Relative content (μg/kg) | | | |
|---|---|---|---|---|
| Odorants | Patriot | HOLL | Tiger | Super |
| Aldehydes | | | | |
| Butanal, 2-methyl | ND | ND | ND | 0.06 ± 0.01 |
| Hexanal | 13.4 ± 0.51^{a} | 4.72 ± 0.29^{b} | 4.74 ± 0.32^{b} | 3.45 ± 0.09^{b} |
| 2-Hexanal, (E)- | 0.46 ± 0.26^{a} | 0.12 ± 0.01^{a} | 0.16 ± 0.02^{a} | 0.11 ± 0.01^{a} |
| Heptanal | 0.75 ± 0.01^{b} | 0.96 ± 0.14^{ab} | 0.99 ± 0.08^{a} | 0.25 ± 0.01^{c} |
| 2-Heptenal, (Z)- | 0.25 ± 0.10 | ND | ND | ND |
| Benzaldehyde | 0.41 ± 0.06^{a} | 0.25 ± 0.02^{b} | 0.24 ± 0.02^{b} | 0.12 ± 0.02^{b} |
| Octanal | 0.43 ± 0.06^{b} | 1.20 ± 0.19^{a} | 0.91 ± 0.07^{a} | 0.36 ± 0.03^{b} |
| 2-Octenal, (E)- | 0.31 ± 0.15 | ND | ND | ND |
| Nonanal | 1.25 ± 0.28^{b} | 3.15 ± 0.41^{a} | 3.10 ± 0.23^{a} | 1.36 ± 0.17^{b} |
| 2-Nonenal, (E)- | 0.09 ± 0.10 | ND | ND | ND |
| 2-Octenal, 2-butyl | 0.13 ± 0.00 | ND | ND | ND |
| Total Aldehydes (11) | 17.5 ± 1.54 | 10.4 ± 0.44 | 10.13 ± 0.66 | 5.72 ± 0.30 |
| Alcohols | | | | |
| 1-Penten-3-ol | ND | 0.10 ± 0.01^{a} | 0.11 ± 0.02^{a} | 0.05 ± 0.01^{a} |
| 1-Pentanol | 0.03 ± 0.00^{b} | 0.10 ± 0.01^{ab} | 0.13 ± 0.02^{a} | 0.06 ± 0.01^{b} |
| 2-Penten-1-ol, (Z) | ND | 0.11 ± 0.02^{a} | 0.13 ± 0.03^{a} | 0.04 ± 0.01^{a} |
| 1-Hexanol | 3.12 ± 0.16^{a} | 1.21 ± 0.46^{b} | 0.97 ± 0.07^{b} | 0.70 ± 0.05^{b} |
| 1-Heptanol | 0.19 ± 0.02^{b} | 0.38 ± 0.01^{a} | 0.35 ± 0.03^{a} | 0.10 ± 0.00^{c} |
| 1-Octen-3-ol | 2.36 ± 0.00^{a} | 0.83 ± 0.05^{d} | 1.99 ± 0.08^{b} | 1.58 ± 0.14^{c} |
| 1-Nonanol | ND | 0.13 ± 0.03^{a} | 0.12 ± 0.05^{a} | ND |
| Total Alcohols (7) | 5.71 ± 0.14 | 2.86 ± 0.50 | 3.79 ± 0.01 | 2.53 ± 0.23 |
| Ketones | | | | |
| 2-Heptanone | 1.63 ± 0.15^{a} | 0.44 ± 0.10^{b} | 0.34 ± 0.06^{b} | 0.14 ± 0.01^{b} |
| 3-Octanone | 0.84 ± 0.27^{a} | 0.17 ± 0.03^{b} | 0.23 ± 0.04^{b} | 0.13 ± 0.00^{b} |
| 2-Octanone | 0.00 ± 0.00^{a} | 0.00 ± 0.00^{a} | ND | 0.12 ± 0.00^{a} |
| 3-Octen-2-one | 0.56 ± 0.21 | ND | ND | ND |
| Acetophenone | ND | ND | ND | 0.02 ± 0.02 |
| 3,5-Octadien-2-one | 0.67 ± 0.04^{a} | 0.54 ± 0.08^{a} | 0.52 ± 0.00^{a} | 0.20 ± 0.01^{b} |
| 2-Nonanone | 0.00 ± 0.00^{b} | 0.00 ± 0.00^{a} | 0.00 ± 0.00^{ab} | 0.00 ± 0.00^{b} |
| 3,5-Octadien-2-one, (E, | 0.34 ± 0.02^{a} | 0.09 ± 0.03^{b} | 0.09 ± 0.02^{b} | ND |
| E)- | | | | |
| 2-Decanone | ND | 0.33 ± 0.10^{a} | 0.21 ± 0.07^{a} | 0.15 ± 0.02^{a} |
| Total ketones (9) | 4.04 ± 0.69 | 1.57 ± 0.07 | 1.39 ± 0.15 | 0.64 ± 0.04 |
| Other Hydrocarbons | | | | |
| Toluene | ND | 0.09 ± 0.02^{a} | 0.08 ± 0.02^{a} | 0.08 ± 0.00^{a} |
| D-Limonene | ND | ND | ND | 0.05 ± 0.02 |
| Furan, 2-pentyl | 2.80 ± 0.52^{a} | 0.49 ± 0.07^{b} | 0.74 ± 0.08^{b} | 0.20 ± 0.03^{b} |
| Ethyl acetate | 0.05 ± 0.01^{b} | 0.13 ± 0.03^{ab} | 0.11 ± 0.01^{ab} | 0.15 ± 0.01^{a} |
| Disulfide, dimethyl | ND | 0.06 ± 0.03^{a} | 0.05 ± 0.03^{a} | 0.05 ± 0.00^{a} |
| Pyridine, 2-pentyl | ND | 0.00 ± 0.00^{a} | 0.00 ± 0.00^{a} | 0.00 ± 0.00^{a} |
| Total compounds (33) | 30.1 ± 2.61^{a} | 15.8 ± 0.02^{b} | 16.3 ± 0.93^{b} | 9.42 ± 0.15^{c} |

TABLE 14

| | | Odor | | | | |
|---|---|---|---|---|---|---|
| Compounds | OTVs | description | Patriot | HOLL | Tiger | Super |
| Hexanal | 4.5 | Green; | 100 | 33.3 | 34 | 48.4 |
| | | grassy | | | | |
| 1-Octen-3-ol | 1 | Mushroom | 79.5 | 26.4 | 64.3 | 100 |
| Nonanal | 1 | Green; fatty | 41.6 | 100 | 100 | 85.9 |
| Octanal | 0.7 | Green; fatty | 20.5 | 54.2 | 42.1 | 32.6 |
| 2-pentyl | 6 | Beany | 15.6 | 2.59 | 4 | 2.13 |
| furan | | | | | | |
| 2-(E)- | 0.25 | Green | 12.1 | ND | ND | ND |
| Nonenal | | | | | | |
| Heptanal | 3 | Green | 8.36 | 10.2 | 10.6 | 5.32 |
| 2-(E)- | 3 | Green | 3.39 | ND | ND | ND |
| Octenal | | | | | | |
| 3-Octen-2- | 6.7 | Earthy | 2.75 | ND | ND | ND |
| one | | | | | | |
| 2-(E)- | 15.2 | Green; | 0.99 | 0.25 | 8 | 0.46 |
| Hexanal | | grassy | | | | |

What is claimed is:

1. A soybean plant, or a progeny, a plant part, or a plant cell thereof, having one or more improved flavor characteristics, comprising a mutant allele of the endogenous FAD2-1A, FAD2-1B, FAD3A, FAD3C, Lox1, Lox2, and Lox3 genes.

2. The soybean plant of claim 1, further comprising a mutant allele of the endogenous FAD3B gene.

3. The soybean plant of claim 1, further comprising a mutant allele of the endogenous RS2, and/or RS3 gene.

4. The soybean plant of claim 1, wherein the mutant allele is a loss-of-function allele, a null allele, or a hypomorphic allele.

5. The soybean plant of claim 1, wherein the endogenous FAD2-1A gene comprises a nucleotide sequence having at least 90%, at least 95%, at least 96%, at least 97%, at least 98%, at least 99%, or 100% sequence identity to SEQ ID NO: 1, wherein the endogenous FAD2-1B gene comprises a nucleotide sequence having at least 90%, at least 95%, at least 96%, at least 97%, at least 98%, at least 99%, or 100% sequence identity to SEQ ID NO: 2, wherein the endogenous FAD3A gene comprises a nucleotide sequence having at least 90%, at least 95%, at least 96%, at least 97%, at least 98%, at least 99%, or 100% sequence identity to SEQ ID NO: 3, wherein the endogenous FAD3C gene comprises a nucleotide sequence having at least 80%, at least 90%, at least 95%, at least 96%, at least 97%, at least 98%, at least 99%, or 100% sequence identity to SEQ ID NO: 4, wherein the endogenous Lox1 gene comprises a nucleotide sequence having at least 90%, at least 95%, at least 96%, at least 97%, at least 98%, at least 99%, or 100% sequence identity to SEQ ID NO: 6, wherein the endogenous Lox2 gene comprises a nucleotide sequence having at least 90%, at least 95%, at least 96%, at least 97%, at least 98%, at least 99%, or 100% sequence identity to SEQ ID NO: 7, or wherein the endogenous Lox3 gene comprises a nucleotide sequence having at least 90%, at least 95%, at least 96%, at least 97%, at least 98%, at least 99%, or 100% sequence identity to SEQ ID NO: 8.

6. The soybean plant of claim 1, wherein the endogenous FAD2-1A gene encodes a polypeptide having at least 90%, at least 95%, at least 96%, at least 97%, at least 98%, at least 99%, or 100% sequence identity to SEQ ID NO: 11, wherein the endogenous FAD2-1B gene encodes a polypeptide having at least 90%, at least 95%, at least 96%, at least 97%, at least 98%, at least 99%, or 100% sequence identity to SEQ ID NO: 12, wherein the endogenous FAD3A gene encodes a polypeptide having at least 90%, at least 95%, at least 96%, at least 97%, at least 98%, at least 99%, or 100% sequence identity to SEQ ID NO: 13, wherein the endogenous FAD3C gene encodes a polypeptide having at least 90%, at least 95%, at least 96%, at least 97%, at least 98%, at least 99%, or 100% sequence identity to SEQ ID NO: 14, wherein the endogenous Lox1 gene encodes a polypeptide having at least 90%, at least 95%, at least 96%, at least 97%, at least 98%, at least 99%, or 100% sequence identity to SEQ ID NO: 16, wherein the endogenous Lox2 gene encodes a polypeptide having at least 90%, at least 95%, at least 96%, at least 97%, at least 98%, at least 99%, or 100% sequence identity to SEQ ID NO: 17, or wherein the endogenous Lox3 gene encodes a polypeptide having at least 90%, at least 95%, at least 96%, at least 97%, at least 98%, at least 99%, or 100% sequence identity to SEQ ID NO: 18.

7. The soybean plant of claim 1, wherein the mutant allele comprises one or more nucleotide modifications within the coding region, non-coding region, regulatory sequence, or untranslated region of the endogenous FAD2-1A, FAD2-1B, FAD3A, FAD3C, Lox1, Lox2, and/or Lox3 gene.

8. The soybean plant of claim 7, wherein the one or more nucleotide modifications reduce expression of the endogenous FAD2-1A, FAD2-1B, FAD3A, FAD3C, Lox1, Lox2, and/or Lox3 gene; reduce transcriptional activity of the polypeptide encoded by the endogenous FAD2-1A, FAD2-1B, FAD3A, FAD3C, Lox1, Lox2, and/or Lox3 gene; generate one or more alternative spliced variants of the endogenous FAD2-1A, FAD2-1B, FAD3A, FAD3C, Lox1, Lox2, and/or Lox3 gene; introduce a frameshift mutation in one or more exons of the endogenous FAD2-1A, FAD2-1B, FAD3A, FAD3C, Lox1, Lox2, and/or Lox3 gene; delete a substantial portion of the endogenous FAD2-1A, FAD2-1B, FAD3A, FAD3C, Lox1, Lox2, and/or Lox3 gene; delete a full-length open reading frame of the endogenous FAD2-1A, FAD2-1B, FAD3A, FAD3C, Lox1, Lox2, and/or Lox3 gene; repress an enhancer motif present within a regulatory region encoding the endogenous FAD2-1A, FAD2-1B, FAD3A, FAD3C, Lox1, Lox2, and/or Lox3 gene; modify one or more nucleotides of a regulatory element operably linked to the endogenous FAD2-1A, FAD2-1B, FAD3A, FAD3C, Lox1, Lox2, and/or Lox3 gene; or any combination thereof.

9. The soybean plant of claim 1, wherein the one or more improved flavor characteristics comprise high oleic acid and/or low linolenic acid relative to a soybean plant without the mutant allele.

10. A seed or an asexual propagate of the soybean plant of claim 1.

11. A method for improving one or more flavor characteristics of soybean seed, the method comprising:
introducing a mutant allele of the endogenous FAD2-1A, FAD2-1B, FAD3A, FAD3C, Lox1, Lox2, and Lox3 genes in a soybean plant.

12. The method of claim 11, further comprising introducing a mutant allele of the endogenous FAD3B gene in the soybean plant.

13. The method of claim 11, further comprising further introducing a mutant allele of the endogenous RS2 and/or RS3 gene in the soybean plant.

14. The method of claim 11, wherein the mutant allele is a loss-of-function allele, a null allele, or a hypomorphic allele.

15. The method of claim 11, wherein the mutant allele is introduced through traditional breeding techniques or gene editing.

16. The method of claim 11, wherein the endogenous FAD2-1A gene comprises a nucleotide sequence having at least 90%, at least 95%, at least 96%, at least 97%, at least 98%, at least 99%, or 100% sequence identity to SEQ ID NO: 1, wherein the endogenous FAD2-1B gene comprises a nucleotide sequence having at least 90%, at least 95%, at least 96%, at least 97%, at least 98%, at least 99%, or 100% sequence identity to SEQ ID NO: 2, wherein the endogenous FAD3A gene comprises a nucleotide sequence having at least 90%, at least 95%, at least 96%, at least 97%, at least 98%, at least 99%, or 100% sequence identity to SEQ ID NO: 3, wherein the endogenous FAD3C gene comprises a nucleotide sequence having at least 80%, at least 90%, at least 95%, at least 96%, at least 97%, at least 98%, at least 99%, or 100% sequence identity to SEQ ID NO: 4, wherein the endogenous Lox1 gene comprises a nucleotide sequence having at least 90%, at least 95%, at least 96%, at least 97%, at least 98%, at least 99%, or 100% sequence identity to SEQ ID NO: 6, wherein the endogenous Lox2 gene comprises a nucleotide sequence having at least 90%, at least 95%, at least 96%, at least 97%, at least 98%, at least 99%, or 100% sequence identity to SEQ ID NO: 7, or wherein the endogenous Lox3 gene comprises a nucleotide sequence having at least 90%, at least 95%, at least 96%, at least 97%, at least 98%, at least 99%, or 100% sequence identity to SEQ ID NO: 8.

17. The method of claim 11, wherein the endogenous FAD2-1A gene encodes a polypeptide having at least 90%, at least 95%, at least 96%, at least 97%, at least 98%, at least 99%, or 100% sequence identity to SEQ ID NO: 11, wherein the endogenous FAD2-1B gene encodes a polypeptide having at least 90%, at least 95%, at least 96%, at least 97%, at least 98%, at least 99%, or 100% sequence identity to SEQ ID NO: 12, wherein the endogenous FAD3A gene encodes a polypeptide having at least 90%, at least 95%, at least 96%, at least 97%, at least 98%, at least 99%, or 100% sequence identity to SEQ ID NO: 13, wherein the endogenous FAD3C gene encodes a polypeptide having at least 90%, at least 95%, at least 96%, at least 97%, at least 98%, at least 99%, or 100% sequence identity to SEQ ID NO: 14, wherein the endogenous Lox1 gene encodes a polypeptide having at least 90%, at least 95%, at least 96%, at least 97%, at least 98%, at least 99%, or 100% sequence identity to SEQ ID NO: 16, wherein the endogenous Lox2 gene encodes a polypeptide having at least 90%, at least 95%, at least 96%, at least 97%, at least 98%, at least 99%, or 100% sequence identity to SEQ ID NO: 17, or wherein the endogenous Lox3 gene encodes a polypeptide having at least 90%, at least 95%, at least 96%, at least 97%, at least 98%, at least 99%, or 100% sequence identity to SEQ ID NO: 18.

18. A commodity plant product prepared from the soybean plant, plant part, or plant cell of claim 1.

19. A method of producing a soybean plant having one or more improved flavor characteristics, the method comprising:
(a) crossing the soybean plant of claim 1 with itself or another soybean plant to produce a seed; and
(b) growing a progeny soybean plant from the seed to produce a soybean plant having one or more improved flavor characteristics.

20. The method of claim 19, further comprising:
(c) crossing the progeny soybean plant with itself or another soybean plant; and
(d) repeating steps (b) and (c) for an additional 0-7 generations to produce a progeny soybean plant of a subsequent generation having one or more improved flavor characteristics.


SEQUENCE LISTING (20 sequences)

SEQ ID NO: 1 - DNA, 1977 bp, Glycine max
  source 1..1977: mol_type = genomic DNA
ttatgacatgtaattgaattttttaattataaaaaataataaaacttaattacgtactataaagagatgctcttgactagaattgtgatctcctagtttcctaaccatatactaatatttgcttgtattgatagcccctccgttcccaagagtataaaactgcatcgaataatacaagccactaggcatggtaaattaaattgtgcctgcacctcgggatatttcatgtggggttcatcatatttgttgaggaaaagaaactcccgaaattgaattatgcatttatatatcctttttcatttctagatttcctgaaggcttaggtgtaggcacctagctagtagctacaatatcagcacttctctctattgataaacaattggctgtaatgccgcagtagaggacgatcacaacatttcgtgctggttactttttgttttatggtcatgatttcactctctctaatctctccattcattttgtagttgtcattatctttagatttttcactacctggtttaaaattgagggattgtagttctgttggtacatattacacattcagcaaaacaactgaaactcaactgaacttgtttatactttgacacagggtctagcaaaggaaacaacaatgggaggtagaggtcgtgtggccaaagtggaagttcaagggaagaagcctctctcaagggttccaaacacaaagccaccattcactgttggccaactcaagaaagcaattccaccacactgctttcagcgctccctcctcacttcattctcctatgttgtttatgacctttcatttgccttcattttctacattgccaccacctacttccacctccttcctcaacccttttccctcattgcatggccaatctattgggttctccaaggttgccttctcactggtgtgtgggtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggcgtgtattggtacaggaacaagtattgatggagcaaccaatgggccatagtgggagttatggaagttttgtcatgtattagtacataattagtagaatgttataaataagtggatttgccgcgtaatgactttgtgtgtattgtgaaacagcttgttgcgatcatggttataatgtaaaaataattctggtattaattacatgtggaaagtgttctgcttatagctttctgcct

SEQ ID NO: 2 - DNA, 3434 bp, Glycine max
  source 1..3434: mol_type = genomic DNA
ggagaagcacttctccttttttttttctctgttgaaattgaaagtgttttccgggaaataaataaaataaattaaaatcttacacactctaggtaggtacttctaatttaatccacactttgactctatatatgttttaaaaataattataatgcgtacttactttctcattatactaaatttaacatcgatgattttattttctgtttctcttctttccacctacatacatcccaaaatttagggtgcaattttaagtttattaacacatgtttttagctgcatgctgcctttgtgtgtgctcaccaaattgcattcttctctttatatgttgtatttgaattttcacaccatatgtaaacaagattacgtacgtgtccatgatcaaatacaaatgctgtcttatactggcaatttgataaacagccgtccattttttctttttctctttaactatatatgctctagaatctctgaagattcctctgccatcgaatttctttcttggtaacaacgtcgtcgttatgttattattttattctatttttattttatcatatatatttcttattttgttcgaagtatgtcatattttgatcgtgacaattagattgtcatgtaggagtaggaatatcactttaaaacattgattagtctgtaggcaatattgtcttctttttcctcctttattaatatattttgtcgaagttttaccacaaggttgattcgctttttttgtccctttctcttgttctttttacctcaggtattttagtctttcatggattataagatcactgagaagtgtatgcatgtaatactaagcaccatagctgttctgcttgaatttatttgtgtgtaaattgtaatgtttcagcgttggctttccctgtagctgctacaatggtactgtatatctattttttgcattgttttcattttttcttttacttaatcttcattgctttgaaattaataaaacaatataatatagtttgaactttgaactattgcctattcatgtaattaacttattcactgactcttattgtttttctggtagaattcattttaaattgaaggataaattaagaggcaatacttgtaaattgacctgtcataattacacaggaccctgttttgtgcctttttgtctctgtctttggttttgcatgttagcctcacacagatatttagtagttgttctgcatacaagcctcacacgtatactaaaccagtggacctcaaagtcatggccttacacctattgcatgcgagtctgtgacacaacccctggtttccatattgcaatgtgctacgccgtcgtccttgtttgtttccatatgtatattgataccatcaaattattatatcatttatatggtctggaccattacgtgtactctttatgacatgtaattgagttttttaattaaaaaaatcaatgaaatttaactacgtagcatcatatagagataattgactagaaatttgatgacttattctttcctaatcatattttcttgtattgatagccccgctgtcccttttaaactcccgagagagtataaaactgcatcgaatattacaagatgcactcttgtcaaatgaagggggggaaatgatactacaagccactaggcatggtatgatgctaaattaaattgtgcctgcaccccaggatatttcatgtgggattcatcatttattgaggaaaactctccaaattgaatcgtgcatttatatttttttttccatttctagatttcttgaaggcttatggtataggcacctacaattatcagcacttctctctattgataaacaattggctgtaataccacagtagagaacgatcacaacattttgtgctggttactttttgttttatggtcatgatttcactctctctaatctgtcacttccctccattcattttgtacttctcatatttttcacttcctggttgaaaattgtagttctcttggtacatactagtattagacattcagcaacaacaactgaactgaacttctttatactttgacacagggtctagcaaaggaaacaataatgggaggtggaggccgtgtggccaaagttgaaattcagcagaagaagcctctctcaagggttccaaacacaaagccaccattcactgttggccaactcaagaaagccattccaccgcactgctttcagcgttccctcctcacttcattgtcctatgttgtttatgacctttcattggctttcattttctacattgccaccacctacttccacctcctccctcaccccttttccctcattgcatggccaatctattgggttctccaaggttgcattcttactggcgtgtgggtgattgctcacgagtgtggtcaccatgccttcagcaagtacccatgggttgatgatgttatgggtttgaccgttcactcagcacttttagtcccttatttctcatggaaaataagccatcgccgccaccactccaacacgggttcccttgaccgtgatgaagtgtttgtcccaaaaccaaaatccaaagttgcatggtacaccaagtacctgaacaaccctctaggaagggctgcttctcttctcatcacactcacaatagggtggcctttgtatttagccttcaatgtctctggcagaccctatgatggttttgctagccactaccacccttatgctcccatatattcaaatcgtgagaggcttttgatctatgtctctgatgttgctttgttttctgtgacttacttgctctaccgtgttgcaactatgaaagggttggtttggctgctatgtgtttatggggtgccattgctcattgtgaacggttttcttgtgaccatcacatatctgcagcacacacactatgccttgcctcactatgattcatcagaatgggattggctgaggggtgctttggcaactatggacagagattatggaattctgaacaaggtgtttcaccacataactgatactcatgtggctcaccatcttttctctacaatgccacattaccatgcaacggaggcaaccaatgcaatgaagccaatattgggtgagtactaccgatttgatgacacaccattttacaaggcactgtggagagaagcaagagagtgcctctatgtggagccagatgaaggaacatccgagaagggcgtgtattggtacaggaacaagtattgatgaaccaagcaatgggccatagtgggagttatggaagttttgtcacttatcacttaattagtagaatgttataaataagtggatttgccgcgtaatgacttgtgtgcattgtgaaacagcttgtagcgatccatggctataatgtaaaaatatgtggaaagtgttctgcttataactttcttccttaaatgcacactccatagaacaatattattggtact

SEQ ID NO: 3 - DNA, 4229 bp, Glycine max
  source 1..4229: mol_type = genomic DNA
ttggtgttatataaatgcactaccccataattgaatttttcaaagatttcattcttcctcttctaggttattacgcaccacccaccacgtatccctgaaaagagagaaaaacacactaagccaaagccaaagcagcaatggttaaagacacaaagcctttagcctatgctgctaataatggataccaaaaggaagcttttgatcccagtgctcctccaccgtttaagattgcagaaatcagagttgcaataccaaaacattgctgggtcaagaatccatggagatccctcagttatgttctcagggatgtgcttgtaattgctgcattgatggctgctgcaagtcacttcaacaactggcttctctggctaatctattggcccattcaaggaacaatgttctgggctctgtttgttcttggacatgattggtaattaattaatttgttgttacttttttgttataatatgaatctcacacactgctttgttatgcctacctcatttcatttggctttagacaacttaaatttgagatctttattatgttttttgcttatatggtaaagtgattcattcttcacattgaattgaacagtggccatggaagcttttcagacagcccttttctaaatagcctggtgggacacatcttgcattcctcaattcttgtgccataccatggatggttagttcatcccggcttttttgtttgtcattggaagttcttttattgattcaatttttatagcgtgttcggaaacgcgtttcagaaaataatgaaatacatcttgaatctgaaagttataacttttagcttcattgtcattgaaagttcttttattaattatatttttattgcgtgtttggaatcccatttgagaaataagaaatcacgtttaaaatgtgaaagttataactattaacttttgactaaacttgaaaaaatcacatttttgatgtggaaccaaatctgatttgagaaccaagttgattttgatggattttgcaggagaattagccacagaactcaccatcaaaatcatggacacattgagaaggatgaatcctgggttccagtatgtgattaactacttcctctatagttatttttgattcaattaaatttatttatttaataagttcaagaaaaaaggaatctttatacttcagataaagctgttcttgaacatttttttttgtcattatcttagttaaccgagaagatttacaagaatctagacaacatgacaagacttgttagattcactgtgccatttccattgtttgtgtatccaatttatttggtgagtgcttttttttttttacttggaagactacaacacattattattattataatatggttcaaatcaatgacttttaatttctttgtgatgtgcactccattttcagttctcaagaagccccggaaaggaaggttctcacttcaatccctacagcaatctgttcccacccagtgagagaaagggaatagcaatatcaacactgtgttgggttaccatgttttctatgcttatctatctctccttcataactagtccagttctattgctcaagctctatggaattccatattgggtaattaaattactcttacattactttttcctctttttttttatgggtcttaactagtatcacaaaaatattggttaaaaaattttaaaaaaatatttattatgtaaatcataaaagaacataaaaaaaatgatgaataacataattttcgtctcttattaaaaatatttttattttaaatttcttaatcaatatatttagaatctggttaacattttttgaatatttcaattctccaattaaaaatttgaaatagtcaccattaattatgtaattgtttgaacacgtgcagatatttgttatgtggctggactttgtcacatacttgcatcaccatggtcatcatcagaaactgccttggtatcgcggcaaggtaacaaaaataaatagaaaatagtgagtgaacacttaaatgttagatactaccttcttcttcttttttttttttttttgaggttaatgctagataatagctagaaagagaaagaaagacaaatataggtaaaaataaataatataacctgggaagaagaaaacataaaaaaagaaataatagagtctacgtaatgtttggatttttgagtgaaatggtgttcacctaccattactcaaagattctgttgtctacgtagtgtttggactttggagtgaaatggtgttcacctaccattactcagattctgttgtgtcccttagttactgtcttatattcttagggtatattctttattttacatccttttcacatcttacttgaaaagatttttaattattcattgaaatattaacgtgacagttaaattaaaataataaaaaattcgttaaaacttcaaataaataagagtgaaaggatcatcatttttcttctttcttttattgcgttattaatcatgcttctcttcttttttttcttcgctttccacccatatcaaattcatgtgaagtatgagaaaatcacgattcaatggaaagctacaggaactttttttgttttgtttttataatcggaattaatttatactccattttttcacaataaatgttacttagtgccttaaagataatatttgaaaaattaaaaaaattattaatacactgtactactatataatatttgacatatatttaacatgattttctattgaaaatttgtatttattattttttaatcaaaacccataaggcattaatttacaagacccatttttcatttatagctttacctgtgatcatttatagctttaagggacttagatgttacaatcttaattacaagtaaatatttatgaaaaacatgtgtcttaccccttaaccttacctcaacaaagaaagtgtgataagtggcaacacacgtgttgcttttttggcccagcaataacacgtgtttttgtggtgtacaaaaatggacaggaatggagttatttaagaggtggtctcacaactgtggatcgtgactatggttggatcaataacattcaccatgacattggcacccatgttattcaccatcttttccctcaaattcctcattatcacctcgttgaagcggtatattttactattattactcacctaaaaagaatgcaattagtacatttgttttatctcttggaagttagtcattttcagttgcatgattgtaatgttctctctatttttaaaccatgttttcacacctacttcgtttaaaataagaatgtggatactattctaatttctattaacttcttttaaaaaataatgtaaaactagtattaaaaaagaggaaatagattacactctactaatactaatagtataaaaaaaattacattgttattttatcacaaataattatatataattaatttttacaatcattatcttaaaagtcatgtatgatatacagtttttacatgctttggtacttattgtaaagttagtgatttattcattatttatgttatataattggcataaatatcatgtaaccagctcactatactataatgggaacttggtggtgaaaggggtttacaaccctcttttctaggtgtaggtgctttgatacttctggtccctttttatatcaatataaattatattttgctgataaaaaaaacattattaatatataatcattaacttctttaaaaaccgtacctaaaactttatattattaaaaagaagattgagatcagcaaaagaaaaaaaaattaacagtcatttgaattcactgcagacacaagcagcaaaatcagttcttggagagtattaccgtgagccagaaagatctgcaccattaccatttcatctaataaagtatttaattcagagtatgagacaagaccacttcgtaagtgacactggagatgtggtttattatcagactgattctctgcaccttcactcgcaccgagactgagtttcaatttttgggttatttattggattctagctactcaaattactttttttttaatgttacgtttttggagttttaacgttttctgaacaacttgcaaattacatgcatagagagacaggaattcatagtgggcctcaatggaatatttatttgaaattagtaaggtggtaattaataaatattgaattgtcagtttcatttgtaatgatttctaccacttg

SEQ ID NO: 4 - DNA, 2836 bp, Glycine max
  source 1..2836: mol_type = genomic DNA
aattcaccaccatctgctctacactcatcttatatatagctatcacccctctccaaagcagcattgtaacagagaaagaaacatttgagcaaaaatggttcaagcacagcctctacaacatgttggtaatggtgcagggaaagaagatcaagcttattttgatccaagtgctccaccacccttcaagattgcaaatatcagagcagcaattccaaaacattgctgggagaagaacacattgagatctctgagttatgttctgagggatgtgttggtagtgactgcattggtagctgcagcaatcggcttcaatagctggttcttctggccactctattggcctgcacaaggcacaatgttttgggcactttttgttcttggacatgattggtaactaattattattacaaattgttatgttatgttatgttatgttgttgtgcctttttctcagtgatgctttagtcatttcatttcacttggttatgcatgattgttcgttcatatgttctgtcatggtgagttctaatttgattgatgcatggaacagtggtcatggaagtttttcaaacagtcctttgttgaacagcattgtgggccacatcttgcactcttcaattcttgtaccataccatggatggtcggttccttttagcaacttttcatgttcactttgtccttaaatttttttttatgtttgttaaaaaatctttggtctgatttaacaacctaaccatttttacaactcatggattttttgcaggagaattagccacaggactcaccatcagaaccatggccatgttgagaaggatgaatcatgggttccggtattactatgagtttgcttgattaatttccacattttttctttcttcttaattttaatcagtggttagatttggttgtgttccaatagaagaaaagggggtatctagagagatgtgaatttcatgaagtggttcatgattatgtgtctttatgcctttatgtcagcttacagagaaagtttacaagaatctagacaacatgacaagaatgatgagattcactcttcctttccccatctttgcataccccttttatttggtgagaccctctttttccagaatgacagcattattttactatatagtacctcaatttttatatttctaaaattttgaattcttgaaattgaaaggaaaggactttattgggtctagcatctcactctctctttgtgatatgaaccatatatttcagtggagcagaagccctggaaaagaaggctctcatttcaacccttacagcaacttgttctctcctggtgagagaagagatgtgctaacttcaactctgtgttggggcatcatgctttctgtgcttctctatctttccctcacaatgggtccactttttatgctcaagctctatggggttccctatttggtaatctcactctcacactttctttatacatcgcacaccagtgtgggttatttgcaacctacaccgaagtaatgccctataattaatggggttaacacatgtccaagtccaatattttgttcacttatttgaacttgaacatgtgtagatcttcgtcatgtggctggatttcgtcacgtacttgcatcatcatggttacaagcagaaactgccttggtaccgtggccaggtatcccatttaacacaatttgtttcattaacattttaagagaatttttttttcaaaatagttttcgaaattaagcaaataccaagcaaattgttagatctacgcttgtacttgttttaaagtcaaattcatgaccaaattgtcctcacaagtccaaaccgtccactattttattttcacctactttatagcccaatttgtcatttggttacttcagaaaagagaaccccatttgtagtaaatatattatttatgaattatggtagtttcaacataaaacatatttatgtgcagttttgccatccttcaaaagaagatagaaacttactccatgttactctgtctatatgtaatttcacaggaatggagttatctaaggggtggtcttacaacagtagatcgcgactatggttggatcaacaacattcaccatgacattggcacccatgttatccatcaccttttccctcaaattccacattatcatttaatcgaagcggtattaattctctatttcacaagaaattattgtatgtctgcctatgtgatctaagtcaattttcacataacacatgatcaaactttcttaattctttcttctaaattgaaaaagtggattatatgtcaattgaaaattggtcaagaccacaaacatgtgatgatctcccaccttacatataataatttctcctattctacaatcaataatccttctatggtcctgaattgttcctttcttttttcattttcttattctttttgttgtcccacaatagactaaagcagcaaaggcagtgctaggaaagtattatcgtgagcctcagaaatctgggccattgccacttcatctaataaagtacttgctccacagcataagtcaggatcacttcgttagcgactctggcgacattgtgtactaccagactgattcccagctccacaaagattcttggacccagtccaactaaagtttttgatgctacatttacctatttcactcttaaatactatttcctatgtaatatgtaatttagaatatgttacctactcaaatcaattaggtgacatgtataagctttcataaattatgctagaaatgcacttacttttcaaagcatgctatgttagaattgaatctacttttcaatacccagatcatcaattagttgacttcta

SEQ ID NO: 5 - DNA, 4226 bp, Glycine max
  source 1..4226: mol_type = genomic DNA
accctgcggttatataaatgcactatcccataagagtatttttcgaagatttccttcttcctattctaggtttttacgcaccacgtatccctgagaaaagagaggaaccacactctctaagccaaagcaaaagcagcagcagcagcaatggttaaagacacaaagcctttagcctatgctgccaataatggataccaacaaaagggttcttcttttgattttgatcctagcgctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattggtaataatttttgtgtttcttactcttttttttttttttttgtttatgatatgaatctcacacattgttctgttatgtcatttcttcttcatttggctttagacaacttaaatttgagatctttattatgtttttgcttatatggtaaagtgattcttcattatttcattcttcattgattgaattgaacagtggccatggaagcttttcagatagccctttgctgaatagcctggtgggacacatcttgcattcctcaattcttgtgccataccatggatggttagttcatactggcttttttgtttgttcatttgtcattgaaaaaaaatcttttgttgattcaattatttttatagtgtgtttggaagcccgtttgagaaaataagaaatcgcatctggaatgtgaaagttataactatttagcttcatctgtcgttgcaagttcttttattggttaaatttttatagcgtgctaggaaacccattcgagaaaataagaaatcacatctggaatgtgaaagttataactgttagcttctgagtaaacgtggaaaaaccacattttggatttggaaccaaattttatttgataaatgacaaccaaattgattttgatggattttgcaggagaattagccacagaactcaccatcaaaaccatggacacattgagaaggatgagtcatgggttccagtatgtgattaattgcttctcctatagttgttcttgattcaattacattttatttatttggtaggtccaagaaaaaagggaatctttatgcttcctgaggctgttcttgaacatggctcttttttatgtgtcattatcttagttaacagagaagatttacaagaatctagacagcatgacaagactcattagattcactgtgccatttccattgtttgtgtatccaatttatttggtgagtgattttttgacttggaagacaacaacacattattattataatatggttcaaaacaatgactttttctttatgatgtgaactccattttttagttttcaagaagccccggaaaggaaggctctcacttcaatccctacagcaatctgtttccacccagtgagagaaaaggaatagcaatatcaacactgtgttgggctaccatgttttctctgcttatctatctctcattcataactagtccacttctagtgctcaagctctatggaattccatattgggtaactaaattactcctacattgttactttttcctccttttttttattatttcaattctccaattggaaatttgaaatagttaccataattatgtaattgtttgatcatgtgcagatatttgttatgtggctggactttgtcacatacttgcatcaccatggtcaccaccagaaactgccttggtaccgcggcaaggtaacaaaaataaatagaaaatagtgggtgaacacttaaatgcgagatagtaatacctaaaaaaagaaaaaaatataggtataataaataatataactttcaaaataaaaagaaatcatagagtctagcgtagtgtttggagtgaaatgatgttcacctaccattactcaaagattttgttgtgtcccttagttcattcttattattttacatatcttacttgaaaagactttttaattattcattgagatcttaaagtgactgttaaattaaaataaaaaacaagtttgttaaaacttcaaataaataagagtgaagggagtgtcatttgtcttctttcttttattgcgttattaatcacgtttctcttctctttttttttttttcttctctgctttccacccattatcaagttcatgtgaagcagtggcggatctatgtaaatgagtggggggcaattgcacccacaagattttattttttatttgtacaggaataataaaataaaactttgcccccataaaaaataaatattttttcttaaaataatgcaaaataaatataagaaataaaaagagaataaattattattaattttattattttgtactttttatttagtttttttagcggttagatttttttttcatgacattatgtaatcttttaaaagcatgtaatatttttattttgtgaaaataaatataaatgatcatattagtctcagaatgtataaactaataataattttatcactaaaagaaattctaatttagtccataaataagtaaaacaagtgacaattatattttatatttacttaatgtgaaataatacttgaacattataataaaacttaatgacaggagatattacatagtgccataaagatattttaaaaaataaaatcattaatacactgtactactatataatattcgatatatatttttaacatgattctcaatagaaaaattgtattgattatattttattagacatgaatttacaagccccgtttttcatttatagctcttacctgtgatctattgttttgcttcgctgtttttgttggtcaagggacttagatgtcacaatattaatactagaagtaaatatttatgaaaacatgtaccttacctcaacaaagaaagtgtggtaagtggcaacacacgtgttgcatttttggcccagcaataacacgtgtttttgtggtgtactaaaatggacaggaatggagttatttaagaggtggcctcaccactgtggatcgtgactatggttggatcaataacattcaccatgacattggcacccatgttatccaccatcttttcccccaaattcctcattatcacctcgttgaagcggtacattttattgcttattcacctaaaaacaatacaattagtacatttgttttatctcttggaagttagtcattttcagttgcatgattctaatgctctctccattcttaaatcatgttttcacacccacttcatttaaaataagaacgtgggtgttattttaatttctattcactaacatgagaaattaacttatttcaagtaataattttaaaatatttttatgctattattttattacaaataattatgtatattaagtttattgattttataataattatattaaaattatatcgatattaatttttgattcactgatagtgttttatattgttagtactgtgcatttattttaaaattggcataaataatatatgtaaccagctcactatactatactgggagcttggtggtgaaaggggttcccaaccctcctttctaggtgtacatgctttgatacttctggtaccttcttatatcaatataaattatattttgctgataaaaaaacatggttaaccattaaattctttttttaaaaaaaaaactgtatctaaactttgtattattaaaaagaagtctgagattaacaataaactaacactcatttgaattcactgcagacacaagcagcaaaaccagttcttggagattactaccgtgagccagaaagatctgcgccattaccatttcatctaataaagtatttaattcagagtatgagacaagaccacttcgtaagtgacactggagatgttgtttattatcagactgattctctgctcctccactcgcaacgagactgagtttcaaactttttgggttattatttattggattctagctactcaaattactttttttttaatgttatgttttttggagtttaacgttttctgaacaacttgcaaattacttgcatagagagacatggaatatttatttgaaattagtaaggtagtaataataaattttgaattgtcagtttc

SEQ ID NO: 6 - DNA, 4270 bp, Glycine max
  source 1..4270: mol_type = genomic DNA
cacaggtcacaggctttttctttttcttcttcttcttcttctttattttctcattttgccaaactaaaatagttgtgttggtagctttggcaaagatgttttcagcaggccataagatcaaagggacagtggtgttgatgcccaagaatgagttggaagttaaccctgatggctcagcagttgacaaccttaatgctttcttgggccgtagtgtctcccttcagctcattagtgctaccaaggctgatggttaattaatttcttcatcgtttcacttttttccatcatcatcatccacaatcatcactttctatattaaaaattaactttaatttctatatacgaaaagtaagtgtaaagagtttaaataatttttgtaaaatagtaatttctgattcaacgatagtatacacattttttacatttttaattcatagagggttttgttttctattagaaatatatcacgtagtaatacataaccaaagatatttatatagtttaaacttactcggtacgtacgtaacatagtattattattatgtgtgtatgatctgtttgtagcacatggaaaaggaaaagttggaaaggatacgttcttggaagggattaatacttcgttaccaactttgggagcaggagagtctgcattcaatattcattttgaatgggacggaagcatgggaatccccggtgcgttttacataaagaactacatgcaagttgagttcttcctcaagagtttgactcttgaagccatttcaaaccaaggaaccatccgctttgtttgcaactcatgggtttataacactaaactttacaaaagcgtgcgcattttctttgccaaccatgtaagctctttagcaattagtagtttaattacaaaagtgtgtgtgtgtgtgaaatgtctaatggcaagttcttcaagtttaagctaagaattaaaaaattcaatttaatgtaatgtagacatatgtacctagtgagacaccagcaccacttgtggagtacagagaagaagaattgaagagtttaagaggaaatggaacgggagagcgcaaggaatatgataggatctatgattatgatgtctacaatgatttgggcaacccagataagagtgaaaagttagctcgtccagttcttggagggtctagcacctttccctaccctcgtaggggaagaactggtcgaggtccaacagttacaggttagtcttctatggttctatataatttaaatgaattgtaacattgactttttataatgttatggtcagcgtagtacaacatattgcttgggtatatgtgacacgttataaattaattttgcactatgattctgattatttttattttttttggatgattaagtttttacacattgttgtttcctaatcaatttcaattcaacagacccgaatactgagaaacaaggcgaagtattttatgttccaagagatgaaaatttaggtcacttgaagtctaaggatgctcttgagattggaacaaaatctttatctcaaattgtccagccggcgttcgaatctgcgttcgatttgaaatccacgccaattgagtttcatagcttccaagatgtgcatgacttgtatgaaggtggaattaagcttcctagagatgtaattagcacaattatccccttaccagtgatcaaagaactctatcgtaccgatggtcaacacatcctcaagtttccacaacctcacgtcgttcaaggtgtgataaataaatataataaaccactcatacttgaaatcttgaattaatcaatgacataataaatataataaaccactgatacttgaaatcttgaattaattaatgacattttggtcatacaaatcgtatcgtattcacgaacaaattcaaaactcattatgttttttatgaaatggttattccatttaataaggattttctttttattattaatttgggtagtgagtcagtctgcatggatgactgatgaagaatttgctagagagatgattgctggtgtaaatccctgcgtaattcgtggtcttgaagtaagttcaaatatttatttataaaatcaaatttgatgtccacgtaaaattaggtagtatatcgccagtaataaactaaaatactaaatggttgttcttctttaattaaatggattgtaggagttccctccaaaaagtaatctggatcctgcaatctatggtgatcaaagcagtaagataacagcagattcccttgatctagatgggtacacaatggacgaggtaaacatgcgaagcgaaggaattgatacaataattttcctcttaaagtaattaattaaaactaattgaattttgtggtacaggcacttggtagtcgaaggttatttatgttagattaccatgatatcttcatgccatatgtgaggcagataaatcagctgaattctgccaagacttatgcgacaaggactatcctttttttgagagaagatggaactttaaagccagtggccatcgaattaagtttgccacattctgctggggatctgtcagctgccgtcagtcaagtcgtcttacctgctaaggaaggtgttgagagcacaatttggctactagccaaagcttatgtcatcgtaaatgactcttgctaccatcaactcatgagccattggtataaattttcattttcattttcattgctatatatcaacatatatagataaattcaatctctaatccctctctctcaagtgtatattaattacttttgatttcttcataggttaaatactcatgcggcgatggagccattcgtcatagcaacacaccgacatcttagcgtgcttcacccaatttacaagcttctgactcctcactatcgtaacaacatgaacatcaacgcacttgccaggcaatctctaattaatgctaatggcataatagagacaacctttttgccctcaaagtattctgtggagatgtcttcggcggtttataagaattgggttttcactgatcaagcactacctgcagatcttatcaagaggtaattaattaagtaaatctctctatatcactagaaaaaaaatgtgacataaaggtttgaaatatataggtatacattttgttacaaagcataatgatggatgcatgtatatattatattttgtttgacttagaggagtggcaattaaggatccatcaaccccacatggagttcgtcttctgatagaggactatccttatgccgctgatggactggagatatgggctgcaattaagacatgggttcaagaatatgtgcccttgtactatgcaagagatgatgatgtcaaaaatgattctgaactccaacattggtggaaagaagctgtagagaaaggccatggtgatttgaaagacaagcatggtggcctaagttgcagacacttgaagaccttgttgaagtttgcctcattatcatatggattgcttcagctctccatgcagccgttaattttggtcagtatccttatggaggtttgataatgaaccgcccaactgcttctagaaggttgcttcctgagaaaggcaccccagaatatgaagaaatgattaataaccatgaaaaggcttatttgagaacaattacatcaaagttgccgactctcattagcctttcagtgatagagatcttgtcgacacatgcttctgatgaggtctaccttggccagagggacaacccacattggacctctgattcaaaagcattacaagcctttcaaaagtttggaaacaagctcaaggaaattgaggaaaaacttgtgaggaggaacaatgatccgagtctgcagggcaatcgacttggcccggttcaactgccatacactttgctttatcctagcagtgaggaagggttaacttttaggggaattccaaatagcatctctatctaagggagcctgtggtttactctaattactctatatatagtgcatgtgtaccttccaataaaaaagatgcaagactagagatccgataaatcttgcatcttatctaatgtttcaattatcttgtgttttaattaatgttgtaattgagctcacagtttggttgtggtttgcaaataataagagcagtgagcactattagtttgtgtttagctcctgtcatcatgtttggtattatacattttgcagattacgtaacacaaatttatgttattagtaaaaaaactgtattgtacgaatattttgtattataagtatataagaaataaaaattattagtattagtaaaaaa

SEQ ID NO: 7 - DNA, 4297 bp, Glycine max
  source 1..4297: mol_type = genomic DNA
acaagtcacaagcttttctttttcttgttctttaattttctgattttagcaaattaaaatagtagtgttattggtaggttttggcaaagatgttttcagttccaggggtgtcgggaatcctgaacagaggaggagggcataagataaaagggacagtggtgttgatgaggaagaatgtgttggacttcaacagcgtggctgatcttactaaaggaaatgttgggggactcataggcaccggcctcaacgttgttggctcaacacttgataacctcactgctttcttgggccgaagtgtcgccctacagctcattagtgctaccaaacctcttggttcatttcttcttccttccacataatcaataacttctatattcaaaattaagtgtttaatctctatactctcattcatttcattcaatgaaaaaaaaatcataagacttttaactaaaattaacctatgtaaagaatcgcaaacaaaaaactatataatattaagtttatttactttttttataatgacaaaaaaaattattgtatatggtgcacaaatttttgtactcttaaaaatatatcactttatacatagccaaacatatttattttgtatagtattaacttatttgggtacgtaccttaataatattattatgtgtgtatgtatggtctgtttgtagcaaatggaaaaggaaaagttggaaaggatacgttcttggaagggattattgtgtcgttaccaactttgggagcaggggagtctgcattcaatattcagtttgaatgggacgaaagcatgggaatccccggtgcgttttacataaagaactacatgcaagttgagttttacctcaagagtttgactcttgaagacgttccaaaccaaggaaccattcgctttgtttgcaactcatgggtttataacactaaactttacaaaagcgtgcgcattttctttgccaaccatgtaagctatttatattacgtacttagctagtagtttataattaaaattcagtatgtatatatatatatagttcatttgtccgtctctaatggcaagatctttaatacgttaatttaatgaattgtagacatatgttccaagtgagacaccagcagcacttgtggggtacagagaagaagaattgaagaatttaagaggagatggaaaaggagagcgcaaagaacatgataggatctatgattatgatgtctacaatgatttgggcaatccagatcacggtgaaaattttgctcgcccaattcttggagggtctagcactcatccctaccctcgtaggggaagaactggtcgatatccaacaaggaaaggttagttttctatatatgcttctatttgtttaagtgaattgatattatatgtgtgaagctaatttaatttattttagcattgttcttttacgatgttatggccagccaggttcaaacaagtgaaagtatctatgcgtacatatttattttttattaaaatgattttatgtatataagttattttgtgctatgattctgatttatttttcctaatcaatttcaacagatcagaattctgagaagccaggcgaagtttatgttccaagagatgaaaattttggtcacttgaagtcctcggattttcttgcatatggaataaaatctttatctcaatatgtcttaccggcgttcgaatctgttttcgatttgaacttcacgccaaatgagtttgatagcttccaagatgtgcgtgacctccacgaaggtggaattaagcttcctacagaagtaattagcacaattatgcccttaccggtggtcaaagaactctttcgtacagatggtgaacaagtcctcaagtttccaccacctcatgtcattcaaggtgaagaatttgctcatacttgaattcttgaatgacactttgctcatacaaatggtatcgtaccatatattcacaaacaaatgcaaattaaacttatgttttttttattaaatatttagttccatttaataatgattttcttcatatatagtgagtaagtctgcatggatgaccgatgaagaatttgcaagagagatggttgcgggtgtaaatccatgcgtaattcgtggtcttcaagtaagtttaaatacttattgcagaaacagattggcaaaccaaatttcgtgtccacattaaattagtctgttgctagctagttataataaccattatatatatatatatatatatatatatatatcatgcggatccggagtcacgtacagtttatcatatatattttgacatgcatgatattcttctttaattggagtataatggattgtaggagtttcctccaaaaagcaatctggatcctacaatctatggtgagcaaaccagtaagataacagcagatgcccttgatctagatggatacacagtggacgaggtaaacatattcaagtctcataatttaaccacaggaagaaggaactttaagtaattaaaagtgcttgtgtatgatctgattacaataaatttaatttgtggcacaggcactagcaagtcggaggttatttatgttagattaccatgatgtattcatgccatatataaggcggataaatcagacatatgccaaggcttatgcgacaaggactatcctttttctgagagaaaatggaaccttaaagccagtggccatcgaattaagtttgccacatcctgctggggacctgtcaggtgctgtcagtcaagtcatcttacctgcaaaagaaggtgttgaaagtacaatttggctactggccaaagcttatgtggtcgtaaatgactcttgctatcatcaactcatgagccattggtatataaaacaattcaattcaatctccatctatgatgtatgttatgtctcaattttattttatttttattttttattttgttcataggttaaatactcatgcggtgattgagccattcatcatagcaacaaaccgacaccttagtgctcttcacccaatttataagcttctaactcctcactaccgtgacaccatgaacatcaacgcacttgctaggcaatctctcattaatgctgatggcataatagagaaatcttttttgccctcaaagcattccgttgagatgtcttcagcggtttataagaattgggttttcactgatcaagcactacctgcagatcttatcaagaggtaattaatctctctaaactctaaacataaaactatgaaatataggtacatagtttgttattgatttatattttgtttgattcagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcttctgatgaggtctaccttggccaaagggacaacccacattggacctctgactcaaaagcattacaagcctttcaaaaatttggaaacaagctcaaagaaattgaggaaaaacttgcaaggaagaacaatgatcaaagtctctccaatcgacttgggccggttcaactgccatacactttgctccatcctaacagtgaggaagggttgacttgcagggggattcctaatagcatctctatctaagggagcatgtggtctactttaattacagtactgtacctaccttccaataaaaaagatgcaaggctagagatccaataaatcttgcatcctatctaatgtttcaattatcttgtgttttaatgctgtaatggagcctacaatttgcaaattaattaagagcaccgagcactattagtttgcgtttagcttctgtcatgtttattaagatatatttcgcagattgtaacacaaaattttgtggcaaaacaagcttttttttttcttattga

SEQ ID NO: 8 - DNA, 4351 bp, Glycine max
  source 1..4351: mol_type = genomic DNA
atcctcacccaaaaaaaaagatcatacatagtagtgttggtgggttgcaaagatgcttgggggtcttctccataggggtcataagataaaagggacagtggtgttgatgcgcaagaatgtgttggacgtgaatagcgtaaccagcgttgggggaattattggtcaaggtctcgacttagttggctcaacactcgatactcttactgccttcttgggccgatccgtgtctctccagcttattagtgctaccaaagctgatggttccttttcttctttcttatttttcctaattacttcatccatgcatgcatgccttcaattctcacctatgttgctcatcaactgatgccaacatctattagtgttttcgtttaacggtgtttcatttcaatctatcctctcacatacttctccgttcaacggagtttgaattcgtgtaaaacataaaccatacacacgctagatgtttataatcatgacgcgtgtcctccttgtgtgtcttcattatttgttatatgagatttgaatatgttattaattattagtatagcatgtgcagcattctcactctcttaccaagctagctacgtttgcacgcacgtctataaatgtttaattttttgtttatgtgaaatgtgaaactaagtcatggtagaacggcttcatattggtttttacttttgttttcactctttcttttctttgggtagccaacggaaaagggaaacttggaaaggctacctttttggaaggtatcattacttcattgccaactttgggagcaggccaatctgcattcaaaattaattttgaatgggatgatgggagtggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccaggtcaagtataattaatgaaacttaaatataacttttaaatcttcaataactttgtttgttttctctagttatttcatttttccaaaaaaaagtttttgtcaatacccgagtgtcaattaatgtggtttattttgaagttgttgaactttaaggcatcaatgaaaaaataaagttaacaaaacatattgcagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaaggttaggcttctatttctatttttaaattactctatcacagactcaaatctgttcttcaattctataatatttatgaggtcttagcataaatgtacttttaatatgtcaagagtcatataactttgaattggtatattgttttagctagaagagttctacatgtacatgttaacataaaggttgactgtttctaatcaagtaatcctaaattatcaaggagtacttagaaacaaccatttcattaaactttaactgtttttcaattatgagtttgaaaatttctgtttttgttgctaataagttctgctattttcattaaacagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaaggtatctcataatattaagtacctgtatctaaaatggcgttacaattaacaacattggagtgctaataatggatatctgaattaatcctatttcactaaggatatttgttattatttgagcagtgagtaagtctgcatggatgactgatgaagaatttgcaagagaaatgcttgctggtgtaaatccaaacttgattcgttgtcttaaggttgagttttctttgcatgtcaagtttgtgattgatatccttgattggcaggatgacccaatggaggaggatcatatatatttccttataatttattattgcaggatttccctccacgaagcaagctagatagccaagtctatggtgatcatactagtcaaataaccaaagaacacctagagcccaacttagaagggctcactgtagatgaggtatataatatgataaacttgtgtttcgaaataaaattttatttgatgaattctgttattttatgtgtctagtttttattttagatgatgtttctatgaataataagagtaaattgtatttggttggaaaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatgaaggtgttgaaagttctatttggctgctagcaaaggcttatgtagttgtgaatgactcgtgctatcatcaacttgtcagccattggtatacattttctcaacgaaataatataaaaataaataaacttatatatatatatctataaaatgattattaatatcttttgatggataataggttaaacactcatgcagttgttgagccattcatcatagcaacaaacaggcatctcagtgttgttcaccctatttataaactccttcaccctcactatcgtgacaccatgaacataaatggccttgctcggttatcactggtcaacgacggtggcgttatagaacaaacatttttgtggggaaggtattctgtggaaatgtctgctgtagtttacaaggattgggtttttacagatcaagcattgcctgctgatcttataaaaaggtaaatcatatatcttacaataggaaacatgaaactcaatttaatttatagttaacaaaaaattgaagacacttttgatgttatataattaatgtcatgtgatatagaggaatggcaattgaggatccatcgtgccctcatggcattcgccttgtgatagaggactacccttatgctgttgatggacttgagatatgggatgctatcaagacatgggtccatgaatacgttttcttgtactacaaatcagatgacacacttagagaagatcctgaactccaagcctgctggaaagaactcgtagaggtgggtcatggagacaagaaaaatgagccatggtggcctaagatgcaaactcgtgaagagctagttgaagcttgcgctatcatcatatggactgcttcagcacttcatgcagctgttaattttggacagtatccctatggaggtttaatcttaaaccgtccaactcttagtaggcgattcatgcctgagaaaggttctgctgagtatgaggagctgaggaagaatccccagaaggcttacttgaagactattacaccaaagtttcagacccttattgacctttctgttatagaaatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcccaacagtatctctatctgaagaggtttgtggttccttaagttttgtggctttgaataaaagatagaagagaaggaatctgaatttcttcatccaacctgcacatggtaatctagtgtgtttgagtgctttcatgtttggctttaataaaataaaagcagttgttttgtaaaatgttcagaatcaataaaagcatttactgctttgctttgtgcgctaacgtta

SEQ ID NO: 9 - DNA, 6459 bp, Glycine max
  source 1..6459: mol_type = genomic DNA
tttgctctggctgtaactagaatgcactaggcacaattaaacaaaaataaattctccttctctatataaacccaccatgtcaccacaccctacccagcaaaaccaaaccatagcaaacctaagcaccaaacctctttctttcaagatccttgaattcagtcccatggctccaagcataagcaaaactgtggaactaaattcatttggtcttgtcaacggtaatttgcctttgtccataaccctagaaggatcaaatttcctcgccaacggccacccttttctcacggaagttcccgaaaacataatagtcaccccttcacccatcgacgccaagagtagtaagaacaacgaggacgacgacgtcgtaggttgcttcgtgggcttccacgcggacgagcccagaagccgacacgtggcttccctggggaagctcagaggaataaaattcatgagcatattccggtttaaggtgtggtggaccactcactgggtcggtagcaacggacacgaactggagcacgagacacagatgatgcttctcgacaaaaacgaccagctcggacgcccctttgtgttgattctcccgatcctccaagcctcgttccgagcctccctgcaacccggtttggatgattacgtggacgtttgcatggagagcgggtcgacacgtgtctgtggctccagcttcgggagctgcttatacgtccacgttggccatgacccgtatcagttgcttagagaagcaactaaagtcgttaggatgcatttggggacgttcaagcttctcgaggagaaaaccgcgccaggtatatactcgccgctaaaaattcatcaccaaaattactactgttttgttttgttggagtttttttttttttttttttatatatataataaggaggtattttagacttttattgaatctctcaactattttttcactctcttttttttacttatatttgactcataagaagcatcgaattgctatcaaattatattcaggcctgcaagctttgagggagtgtgtttatgtcatataatagaaatagggtatataaataaaggagaatcctaactttttttggttgacataaatttaaatttaaatcctaaaacacgagtaatttataattttaaggattaaatatgtttttagtttttttattttggttgaatgttatttttagttttcttttatactgtctaatttagttcataaagtttataaaaaaacgtctttttaattcctctttacaacttaaatttttaaaaaatggtgcaaaaattaggcagtaggtctaggaactaaaaataagattatataaaaactaaaaacacaatattcactaatcttaatgagattttttttaaaaaaatagtaaaaatttattttgtaaactgatttattttatattgtagtgatcatagacaagtttggttggtgtacatgggacgcgttttacttgaaggtgcatccctcaggtgtgtgggaaggggtgaaagggttggtggagggagggtgccctccagggatggtcctaatcgacgacgggtggcaagccatttgtcacgacgaggaccccataacggaccaagagggtatgaagcgaacctccgcaggggagcaaatgccatgcaggttggtgaagttggaggaaaattacaagttcagacagtattgtagtggaaaggattctgagaagggtatgggtgcctttgttagggacttgaaggaacagtttaggagcgtggagcaggtgtatgtgtggcacgcgctttgtgggtattggggtggggtcagacccaaggttccgggcatgccccaggctaaggttgtcactccgaagctgtccaatggactaaaattgacaatgaaggatttagcggtggataagatcgtcagtaacggagttggactggtgccaccacacctggctcaccttttgtacgaggggctccactcccgtttggaatctgcgggtattgacggtgttaaggttgacgttatacacgtaagtctctactctgcatttttgttttctttttgacagtattgtctaaccttttaacttaattatttagatgaaaattactcattttgattttgattttaatatggtgtttataggcttcagcaaaaaaaaaaaaaatttggtgtttatagattagtaatacatatagttatagattatagatgcctgtggaagaatttaagaattaattgatgatcaaattgactacagtttagttactacagactacagagtaccttagccatgtttatgttctctgtagtgtatgtttttagtttccaaggtaacattttgaactaaatatcagaattacttcgtaagtttaatttgttgtctttggctacatttattcttgttttctagtttgctacgatacttgtacacggcaagccatatttacaaatatttgtatgtggtgcttaaaaatttgaaaggcaaattattcagtgaagtgtgtaaaccgaaagtgaatattttctttattggtgtatattaaaaaaactaagaatttttatagtattgatgaattaaaaaatttagattaaaaaactaataaaaaataatgttgatgaatttaaaaatgtaattattgtataattcgtttcctaggttggagagaatattgcttaagttatataaagtaaataattagaccaaaatttgtttgacacaagtaatttgacacttgcacacttgggaggcttaatcaagatttgagtttcaattttcaatttgagtatagagtcataacgagagatgctactttattacaaaatgaaatgagttaacccggtgggagagaaatgagttggatctcaaataaaataccctagatatctcttggttacatcaaaagaatttttctttaattgtgacatataatggtaagatctatttaagtggtgtaaaaagaaaaaaaaagagttgctaagttaaagatctctaatatttaatataataatatataccaatagttttaaattagaaaaatgatatatgccttattgtttggtatataaaatcaaaaggaaaaaataaaaataaaagagataaagtgaaagaaagatctaataattaataaataatacaatgtcttaaaaagatatataaaaaataatataaaaataaatatattatataagtatataaattattattactaggggaaagagatccacgtctttctaaaaagaacttctttttttaaataaaacacaaaatgaaaaaggaaaagaacgagagaaagttgaaacaaaaatggaagtgtgcaaatgtgcaattaactttataaaatgatatatagttaaagatgatttaatttttttaaaaaaagtaaaaaccaaaaacttcgcattgtctttattattaattatagattatttagattaagaaaaataaatacaaatacagttttctatatgttagtgaactttgttgtaaaaggttacatgttactaaatacacactagatgctctcattatgtaaatatttgcaataaactacatgttctcatagtgatattttctttaatactctatataatttgtttaaatttattagaaattagtaattttaatgagaagaactcatcaaaatggatgatttttaataaatttcaataaaaatggttaagaaaaagagtaagtattgttagcatttcttattttgaaaatataatagacgttaggttagcaacatcagcacgttcaatattctttctgtatttaagtgttagtctaataggaccaaccatctgcatatatacactacggttgtcataattgcagggttgaaacttgaaagccaaattctctcaactaaaaatgatttatcatgagattgagaagtgtctcttcccttggcacttgtatccttcgtaggaccgcatcaagtgagtatacgaggatcttgctaaattaatattacaactcacaagtatcaacgaggtttccaacttcgtttgatacttggaggtatcattttactataaaaaaatgccatttactaataatttaatgttgggtttgagaatcaagttggatcataatcagctatgaagcccaagactctataaagctagttctacatattaattggcctcaagtggtggctatacacaacggatttttcaatatttctattattatagcaaaacactaaactaaacatagatcttgattaaccatgattacttagatgattttcaagtttaatatttcgtgatttgcatttttgcagttgctcgagatgctatccgaggaatacggtggccgtgttgagctagccaaagcttattacaaagcgctcactgcttcggtgaagaagcatttcaaaggcaatggggtcattgcgagcatggagcattgtaatgacttctttctccttggtaccgaagccatagcccttgggcgcgtaggtaataatcattccaaattgcagcccttataataaacaataacattagtagtcaaagtattttaacttgattggttgaatattatctctgagttattgtaaattttgtaaattttttaatagtgtctttaattctcacggaaaaaaatatattaatagatctttccaaaacatcaaaactttaacatacacaaattttttggagtttaattatcatgtgtcttacacctaattaatcaaaaatcatgtttgacataacttttaaaaaatttgtattaaaagtcaataaatacgatagtttgagattgaacacaaagtaaaattagtgtcaatgcattgtctattttctgaattctccatcaaattttctaacccataacaatacataaattcaggagatgatttttggtgcactgatccctctggagatccaaatggcacgtattggctccaagggtgtcacatggtgcactgtgcctacaacagcttgtggatggggaattttattcagccggattgggacatgttccagtccactcacccttgtgccgaattccatgcagcctctagggccatctctggtggaccagtttacgttagtgattgtgttggaaagcacaacttcaagttgctcaagagcctcgctttgcctgatgggacgattttgcgttgtcaacactatgcactccccacacgagactgtttgtttgaagaccccttgcatgatgggaagacaatgctcaaaatttggaatctcaacaaagtaagctaccctcttttttaaattatttttttctttaatccggtaaattttttgtcagtgttagggactaatcatctgagatgaaaaaaaacaattcaagaggtcggctgggaattaaacttttgatcacatatttaagaagaaattcttacatgatctagactttaaactaccatgattgcaaacaatctttgtccctattgattatttttttaattggcaaatgttagttttgttagaaatgtcagtttggaagatttgatatcctttcatttttccttcttctttcatctttctttcaccactgaaccaacctcgtatctttacatcttattaaattttattttatttttaatcgacaaatgttaattattattattgaattttattaattgttttgagtgaaaattagttgaaaccatgaaaaaatagtgatcatgcaacttctaatattttttttcatgttatgaagcatgcttatttgtcaattatatcagatcaattaattttttaatcacaccataatgacattggaattattaataacctttttatatttgttttgttacagtatacaggtgttttgggtctatttaattgccaaggaggtgggtggtgtcccgtaactaggagaaacaagagtgcctctgaattttcacaaactgtgacatgcttagcgagtcctcaagacattgaatggagcaatgggaaaagcccaatatgcataaaagggatgaatgtgtttgctgtatatttgttcaaggaccacaaactaaagctcatgaaggcatcagagaaattggaagtttcacttgagccatttacttttgagctattgacagtgtctccagtgattgtgctgtcaaaaaagttaattcaatttgctccaattggattagtgaacatgcttaacactggtggtgccattcagtccatggagtttgacaaccacatagatgtggtcaaaattggggttaggggttgtggggagatgaaggtgtttgcatcagagaaaccagttagttgcaaactagatggggtagttgtaaaatttgattatgaggataaaatgctgagagtgcaagttccctggcctagtgcttcaaaattgtcaatggttgagtttttattttgatccctgaaggtgaatttgggatactatgatgtttgactctctttttaagtaataagagtcatatttttctgttgtaggttttagtttcccatgcgtattctttgtccaa

SEQ ID NO: 10 - DNA, 4631 bp, Glycine max
  source 1..4631: mol_type = genomic DNA
ttgggagctttgtcttagtattatttcagcagaaatcaaagacattgaatctcacaaaaaatgggtccaagctcgaagaaagcttcacctaaatcaggtgtgacaaagcacatgaagggcttcagcctctgcaactcaaccctaaaagtaaatgggcaagtcatcctctcccaagtccccaagaacgtaaccctcaccccatgcacctacgacactcacaccaccggatgcttcctcggtttccacgccacctccccaaaatcccgccacgtggcacccttaggacagcttaaaaacataagcttcacttccatcttccggttcaaggtttggtggaccactctctggaccggctccaacggccgcgacctggaaaccgaaacccaattcctcatgctccaatcccacccttatgttctcttcctacccatcctccaacccccatttcgcgcctcgctgcagcctcactcagacgacaacgttgcggtgtgtgtggagagcggctccagccacgtaacagcctcatcattcgacactgtcgtctacttgcacgcaggggacaaccccttcacgctggtcaaggaagccatgcgcgtcgtccgggcccacttggggagcttcaagcttctggaagagaaaaccgttccgggaatggtggacaagttcggttggtgcacgtgggacgccttttacctgacggtgcaccctgagggcgtcagagagggcgtgaagggcctggttgacggcggttgtcctccgggattcgtcctgatcgacgacggctggcagtgcatcagccacgattccgatccggagaaggaggggatgaatcagacggtggccggggagcaaatgccctgcaggtcagtcacgcgcttgagagaaattaaagaaactttttaaataataatataataacaatgctcttgctaacttaatgggaataacaatgcttttgcaaatatgtattatatgtgtggttggtgcacaggttgattagttacgaggagaattacaagtttaggagctataaggaagggaaggggttgaaggggtttgtgagagaattgaaggaggagtttgggtcggtggagtacgtgtacgtgtggcacgcgctgtgcgggtattggggaggggtgaggccgggggtggcggggatggcggaggcggcggtggagaagccaaagctgacggaggggttgaagggaacgatggaggatctggcggtggacaagattgtgaataatggggtcggggtggtgccgccggagctggtgggggaaatgtacgagcgccttcacgcgcacttggagagtgcgggtattgatggggtcaaagttgatgtcatccacgtaagttattttctctttagcatggtatagtatatattcaatttctgaccatactttttaaaaaaaaaaaataaaaataatgtttctttgccctttaatctgcatcaatggatatttgttttctttcttgttaacgactaacgtttaaccacagaaaaaacataccatatttcgtggtggaaaaatcctgctacatattttcgtttgcacaacaacttaattaacatcttatgtgttctttctatttctacttacaagatatgattaactaacttacgtttatttaaaaagttggttaatttggttattagtattaaatttgtttataattataatatttttaaaattttctatttattaataaaattttctataattgtaaataacgacggaagggtatttattatgtaaatttttgtataattgaaagtgaagaaaagttaaactgttttaacttttttattatttgctacacaaagggtgtgacaattttggtttgtttgactttgataacagtcattgtcggttcgtttcactttcacttaaaatttggtcggcacgttatttctttttaatttgaagttttgcccttctctgtttatttatttattttactttttcttctttcaaacttttttaatgtcagcatttatcttttggaccagtcactgttagcatttcttgtcaaatgcaaattgccatgcatgatgaacttttgtttatgctttctgttgggtaatctttgatttccttccttttttggttggcatgtgtttctttgtatggatgctcacctctgatgtgcaaaacagttgctagaaatggtgtgtgagaaatatggagggcgagtggatatggcgaaagcatattacaaagctctcactgcttccgtgagaaaacattttaagggcaacggcgtcattgccagcatggagcattgcaacgatttcatgttgctgggaactgaagcaatatcccttggtcgtgttggtatgtgacgtgtaccttgttagcacaatacaaaccaattttcttacctcaattaatagtacaaaacacaacatcataaaagtatatatattatgcatcttactaactaatgtcgaaattaattaagtaactaaaaagagaaaatatttattataaaaatcataaaaagaacgtaaaaagaatcataaacaatttaattttacatattctaataaaaaatatttatctttttagtttcttagttaatgctttaagaacactgctagcatttgccatatattattaggacaaaattcaagtgttgttgttgttccatcagaattgaagtttcaactcgaaaatctatgcgatctctaatagaaacatttatcaaggtgaaatttcaattctaattagagaacaacactcaaagtacctaagaaattgcattaagttttatccatattattactatttggaattaagttcctttgatctgatgtattagtataacttggattgaaacaggggatgatttctggtgcactgacccttatggtgatccaaatggtacattttggctacaagggtgtcacatggtgcattgtgcatacaacagcttgtggatgggcaatttcatccacccagattgggacatgttccaatctactcatccttgtgctgccttccatgctgcctcaagagccatatctggtggccccatttacatcagtgacacagttgggaaccacaactttgagctgcttaagaccttggccttgccagatgggtccatcctcagatgtgagcactatgcactcccaaccagggactgtctctttgctgaccctctccatgatggcaaaacaatgctcaagatatggaacctcaacaaggtcataattcaattcatcctctccttttcaatcatatgcattattattttgtgtctatatatggctaaagcattatttaagcacaaatcaactatctgatatagtatccctttagactctcttcggataaccttatatatgagcacttgagaaaaaaaaaatgtaaagttaaaatgaattgactgtctcttgtaggtcaaaatcaacttatacacttaatttttataaaagttttctcctctaagttttctcctctaacttttccaaaagctcaagtgtatgagttaattttagtttacaggaaaaattacttattttctttctcttatggagaagtttatcagcaaaggactttaatcttaacaatgaagaaaacgaacaattgagtttttaccaagttacttacaaatatttgctatgtttctataatgatattgcttaaaagcaaattgatcaattaccaatgtcaattggaaaatgcattttacactataaagtgaaacaatttttgctatactatttgatttttcggcaatatatctatgcgtgtataaagaagataaaatagtgagacatagtattaattatttgatgttcaaataatgaaaatggtgtctaatactactactgagactagagcctaacgatattattgtgattttgctttttgatacagtacactggagttcttggggtgtttaactgccagggaggaggttggttccgtgagattaggtccaacaaatgtgctgctgagttttctcatagggtatcaaccaagaccaatatcaaagacattgaatgggatagtggaaagaatccaatttccattgaaggggtgcaacttttcgcttcgtatttcagccaagccaagaaactcatcctctcagcaccatctgatgacagtgaagagatttccttggagccattcaatttcgagcttataacagtttcccctgtgactgtcttgcctggcaagtcagtgaagtttgctcctattggtttggtgaatatgctaaacactggtggagcagtccagtctttagcttttgatgagggtcagaatttggttgaagttggtttaagaggcactggggagatgagagtctatgcctcagagaaaccaagaacttgtagaattgatggcaaagaagttgattttgaatatgaagggtctatggtcaacattcaagtaccatggcctggttcttcaaaattgtccactgttcagtatgtattttaagcctagaagtgatttctttttaacttttgaatggttgtccacagtgaatgatgtgaaagggctttttctcccttactaccgagtgaaataatagagtgatctaataattgtatcaagagctagattcctttttattcaatgaaagccagtttttcttgagtgtacatctgt

SEQ ID NO: 11 - protein, 387 aa, Glycine max
MGLAKETTMGGRGRVAKVEVQGKKPLSRVPNTKPPFTVGQLKKAIPPHCFQRSLLTSFSYVVYDLSFAFIFYIATTYFHLLPQPFSLIAWPIYWVLQGCLLTGVWVIAHECGHHAFSKYQWVDDVVGLTLHSTLLVPYFSWKISHRRHHSNTGSLDRDEVFVPKPKSKVAWFSKYLNNPLGRAVSLLVTLTIGWPMYLAFNVSGRPYDSFASHYHPYAPIYSNRERLLIYVSDVALFSVTYSLYRVATLKGLVWLLCVYGVPLLIVNGFLVTITYLQHTHFALPHYDSSEWDWLKGALATMDRDYGILNKVFHHITDTHVAHHLFSTMPHYHAMEATNAIKPILGEYYQFDDTPFYKALWREARECLYVEPDEGTSEKGVYWYRNKY

SEQ ID NO: 12 - protein, 387 aa, Glycine max
MGLAKETIMGGGGRVAKVEIQQKKPLSRVPNTKPPFTVGQLKKAIPPHCFQRSLLTSLSYVVYDLSLAFIFYIATTYFHLLPHPFSLIAWPIYWVLQGCILTGVWVIAHECGHHAFSKYPWVDDVMGLTVHSALLVPYFSWKISHRRHHSNTGSLDRDEVFVPKPKSKVAWYTKYLNNPLGRAASLLITLTIGWPLYLAFNVSGRPYDGFASHYHPYAPIYSNRERLLIYVSDVALFSVTYLLYRVATMKGLVWLLCVYGVPLLIVNGFLVTITYLQHTHYALPHYDSSEWDWLRGALATMDRDYGILNKVFHHITDTHVAHHLFSTMPHYHATEATNAMKPILGEYYRFDDTPFYKALWREARECLYVEPDEGTSEKGVYWYRNKY

SEQ ID NO: 13 - protein, 376 aa, Glycine max
MVKDTKPLAYAANNGYQKEAFDPSAPPPFKIAEIRVAIPKHCWVKNPWRSLSYVLRDVLVIAALMAAASHFNNWLLWLIYWPIQGTMFWALFVLGHDCGHGSFSDSPFLNSLVGHILHSSILVPYHGWRISHRTHHQNHGHIEKDESWVPLTEKIYKNLDNMTRLVRFTVPFPLFVYPIYLFSRSPGKEGSHFNPYSNLFPPSERKGIAISTLCWVTMFSMLIYLSFITSPVLLLKLYGIPYWIFVMWLDFVTYLHHHGHHQKLPWYRGKEWSYLRGGLTTVDRDYGWINNIHHDIGTHVIHHLFPQIPHYHLVEATQAAKSVLGEYYREPERSAPLPFHLIKYLIQSMRQDHFVSDTGDVVYYQTDSLHLHSHRD

SEQ ID NO: 14 - protein, 380 aa, Glycine max
MVQAQPLQHVGNGAGKEDQAYFDPSAPPPFKIANIRAAIPKHCWEKNTLRSLSYVLRDVLVVTALVAAAIGFNSWFFWPLYWPAQGTMFWALFVLGHDCGHGSFSNSPLLNSIVGHILHSSILVPYHGWRISHRTHHQNHGHVEKDESWVPLTEKVYKNLDNMTRMMRFTLPFPIFAYPFYLWSRSPGKEGSHFNPYSNLFSPGERRDVLTSTLCWGIMLSVLLYLSLTMGPLFMLKLYGVPYLIFVMWLDFVTYLHHHGYKQKLPWYRGQEWSYLRGGLTTVDRDYGWINNIHHDIGTHVIHHLFPQIPHYHLIEATKAAKAVLGKYYREPQKSGPLPLHLIKYLLHSISQDHFVSDSGDIVYYQTDSQLHKDSWTQSN

SEQ ID NO: 15 - protein, 380 aa, Glycine max
MVKDTKPLAYAANNGYQQKGSSFDFDPSAPPPFKIAEIRASIPKHCWVKNPWRSLSYVLRDVLVIAALVAAAIHFDNWLLWLIYCPIQGTMFWALFVLGHDCGHGSFSDSPLLNSLVGHILHSSILVPYHGWRISHRTHHQNHGHIEKDESWVPLTEKIYKNLDSMTRLIRFTVPFPLFVYPIYLFSRSPGKEGSHFNPYSNLFPPSERKGIAISTLCWATMFSLLIYLSFITSPLLVLKLYGIPYWIFVMWLDFVTYLHHHGHHQKLPWYRGKEWSYLRGGLTTVDRDYGWINNIHHDIGTHVIHHLFPQIPHYHLVEATQAAKPVLGDYYREPERSAPLPFHLIKYLIQSMRQDHFVSDTGDVVYYQTDSLLLHSQRD

SEQ ID NO: 16 - protein, 826 aa, Glycine max
MFSAGHKIKGTVVLMPKNELEVNPDGSAVDNLNAFLGRSVSLQLISATKADAHGKGKVGKDTFLEGINTSLPTLGAGESAFNIHFEWDGSMGIPGAFYIKNYMQVEFFLKSLTLEAISNQGTIRFVCNSWVYNTKLYKSVRIFFANHTYVPSETPAPLVEYREEELKSLRGNGTGERKEYDRIYDYDVYNDLGNPDKSEKLARPVLGGSSTFPYPRRGRTGRGPTVTDPNTEKQGEVFYVPRDENLGHLKSKDALEIGTKSLSQIVQPAFESAFDLKSTPIEFHSFQDVHDLYEGGIKLPRDVISTIIPLPVIKELYRTDGQHILKFPQPHVVQVSQSAWMTDEEFAREMIAGVNPCVIRGLEEFPPKSNLDPAIYGDQSSKITADSLDLDGYTMDEALGSRRLFMLDYHDIFMPYVRQINQLNSAKTYATRTILFLREDGTLKPVAIELSLPHSAGDLSAAVSQVVLPAKEGVESTIWLLAKAYVIVNDSCYHQLMSHWLNTHAAMEPFVIATHRHLSVLHPIYKLLTPHYRNNMNINALARQSLINANGIIETTFLPSKYSVEMSSAVYKNWVFTDQALPADLIKRGVAIKDPSTPHGVRLLIEDYPYAADGLEIWAAIKTWVQEYVPLYYARDDDVKNDSELQHWWKEAVEKGHGDLKDKHVCLIIIWIASALHAAVNFGQYPYGGLIMNRPTASRRLLPEKGTPEYEEMINNHEKAYLRTITSKLPTLISLSVIEILSTHASDEVYLGQRDNPHWTSDSKALQAFQKFGNKLKEIEEKLVRRNNDPSLQGNRLGPVQLPYTLLYPSSEEGLTFRGIPNSISI

SEQ ID NO: 17 - protein, 866 aa, Glycine max
MFSVPGVSGILNRGGGHKIKGTVVLMRKNVLDFNSVADLTKGNVGGLIGTGLNVVGSTLDNLTAFLGRSVALQLISATKPLANGKGKVGKDTFLEGIIVSLPTLGAGESAFNIQFEWDESMGIPGAFYIKNYMQVEFYLKSLTLEDVPNQGTIRFVCNSWVYNTKLYKSVRIFFANHTYVPSETPAALVGYREEELKNLRGDGKGERKEHDRIYDYDVYNDLGNPDHGENFARPILGGSSTHPYPRRGRTGRYPTRKDQNSEKPGEVYVPRDENFGHLKSSDFLAYGIKSLSQYVLPAFESVFDLNFTPNEFDSFQDVRDLHEGGIKLPTEVISTIMPLPVVKELFRTDGEQVLKFPPPHVIQVSKSAWMTDEEFAREMVAGVNPCVIRGLQEFPPKSNLDPTIYGEQTSKITADALDLDGYTVDEALASRRLFMLDYHDVFMPYIRRINQTYAKAYATRTILFLRENGTLKPVAIELSLPHPAGDLSGAVSQVILPAKEGVESTIWLLAKAYVVVNDSCYHQLMSHWLNTHAVIEPFIIATNRHLSALHPIYKLLTPHYRDTMNINALARQSLINADGIIEKSFLPSKHSVEMSSAVYKNWVFTDQALPADLIKRGVAIKDPSAPHGLRLLIEDYPYAVDGLEIWAAIKTWVQEYVSLYYARDDDVKPDSELQQWWKEAVEKGHGDLKDKPWWPKLQTIEELVEICTIIIWTASALHAAVNFGQYPYGGFILNRPTSSRRLLPEKGTPEYEEMVKSHQKAYLRTITSKFQTLVDLSVIEILSRHASDEVYLGQRDNPHWTSDSKALQAFQKFGNKLKEIEEKLARKNNDQSLSNRLGPVQLPYTLLHPNSEEGLTCRGIPNSISI

SEQ ID NO: 18 - protein, 857 aa, Glycine max
MLGGLLHRGHKIKGTVVLMRKNVLDVNSVTSVGGIIGQGLDLVGSTLDTLTAFLGRSVSLQLISATKADANGKGKLGKATFLEGIITSLPTLGAGQSAFKINFEWDDGSGIPGAFYIKNFMQTEFFLVSLTLEDIPNHGSIHFVCNSWIYNAKLFKSDRIFFANQTYLPSETPAPLVKYREEELHNLRGDGTGERKEWERIYDYDVYNDLGDPDKGENHARPVLGGNDTFPYPRRGRTGRKPTRKDPNSESRSNDVYLPRDEAFGHLKSSDFLTYGLKSVSQNVLPLLQSAFDLNFTPREFDSFDEVHGLYSGGIKLPTDIISKISPLPVLKEIFRTDGEQALKFPPPKVIQVSKSAWMTDEEFAREMLAGVNPNLIRCLKDFPPRSKLDSQVYGDHTSQITKEHLEPNLEGLTVDEAIQNKRLFLLDHHDPIMPYLRRINATSTKAYATRTILFLKNDGTLRPLAIELSLPHPQGDQSGAFSQVFLPADEGVESSIWLLAKAYVVVNDSCYHQLVSHWLNTHAVVEPFIIATNRHLSVVHPIYKLLHPHYRDTMNINGLARLSLVNDGGVIEQTFLWGRYSVEMSAVVYKDWVFTDQALPADLIKRGMAIEDPSCPHGIRLVIEDYPYAVDGLEIWDAIKTWVHEYVFLYYKSDDTLREDPELQACWKELVEVGHGDKKNEPWWPKMQTREELVEACAIIIWTASALHAAVNFGQYPYGGLILNRPTLSRRFMPEKGSAEYEELRKNPQKAYLKTITPKFQTLIDLSVIEILSRHASDEVYLGERDNPNWTSDTRALEAFKRFGNKLAQIENKLSERNNDEKLRNRCGPVQMPYTLLLPSSKEGLTFRGIPNSISI

SEQ ID NO: 19 - protein, 810 aa, Glycine max
MSPHPTQQNQTIANLSTKPLSFKILEFSPMAPSISKTVELNSFGLVNGNLPLSITLEGSNFLANGHPFLTEVPENIIVTPSPIDAKSSKNNEDDDVVGCFVGFHADEPRSRHVASLGKLRGIKFMSIFRFKVWWTTHWVGSNGHELEHETQMMLLDKNDQLGRPFVLILPILQASFRASLQPGLDDYVDVCMESGSTRVCGSSFGSCLYVHVGHDPYQLLREATKVVRMHLGTFKLLEEKTAPVIIDKFGWCTWDAFYLKVHPSGVWEGVKGLVEGGCPPGMVLIDDGWQAICHDEDPITDQEGMKRTSAGEQMPCRLVKLEENYKFRQYCSGKDSEKGMGAFVRDLKEQFRSVEQVYVWHALCGYWGGVRPKVPGMPQAKVVTPKLSNGLKLTMKDLAVDKIVSNGVGLVPPHLAHLLYEGLHSRLESAGIDGVKVDVIHLLEMLSEEYGGRVELAKAYYKALTASVKKHFKGNGVIASMEHCNDFFLLGTEAIALGRVGDDFWCTDPSGDPNGTYWLQGCHMVHCAYNSLWMGNFIQPDWDMFQSTHPCAEFHAASRAISGGPVYVSDCVGKHNFKLLKSLALPDGTILRCQHYALPTRDCLFEDPLHDGKTMLKIWNLNKYTGVLGLFNCQGGGWCPVTRRNKSASEFSQTVTCLASPQDIEWSNGKSPICIKGMNVFAVYLFKDHKLKLMKASEKLEVSLEPFTFELLTVSPVIVLSKKLIQFAPIGLVNMLNTGGAIQSMEFDNHIDVVKIGVRGCGEMKVFASEKPVSCKLDGVVVKFDYEDKMLRVQVPWPSASKLSMVEFLF

SEQ ID NO: 20 - protein, 758 aa, Glycine max
MGPSSKKASPKSGVTKHMKGFSLCNSTLKVNGQVILSQVPKNVTLTPCTYDTHTTGCFLGFHATSPKSRHVAPLGQLKNISFTSIFRFKVWWTTLWTGSNGRDLETETQFLMLQSHPYVLFLPILQPPFRASLQPHSDDNVAVCVESGSSHVTASSFDTVVYLHAGDNPFTLVKEAMRVVRAHLGSFKLLEEKTVPGMVDKFGWCTWDAFYLTVHPEGVREGVKGLVDGGCPPGFVLIDDGWQCISHDSDPEKEGMNQTVAGEQMPCRLISYEENYKFRSYKEGKGLKGFVRELKEEFGSVEYVYVWHALCGYWGGVRPGVAGMAEAAVEKPKLTEGLKGTMEDLAVDKIVNNGVGVVPPELVGEMYERLHAHLESAGIDGVKVDVIHLLEMVCEKYGGRVDMAKAYYKALTASVRKHFKGNGVIASMEHCNDFMLLGTEAISLGRVGDDFWCTDPYGDPNGTFWLQGCHMVHCAYNSLWMGNFIHPDWDMFQSTHPCAAFHAASRAISGGPIYISDTVGNHNFELLKTLALPDGSILRCEHYALPTRDCLFADPLHDGKTMLKIWNLNKYTGVLGVFNCQGGGWFREIRSNKCAAEFSHRVSTKTNIKDIEWDSGKNPISIEGVQLFASYFSQAKKLILSAPSDDSEEISLEPFNFELITVSPVTVLPGKSVKFAPIGLVNMLNTGGAVQSLAFDEGQNLVEVGLRGTGEMRVYASEKPRTCRIDGKEVDFEYEGSMVNIQVPWPGSSKLSTVQYVF